(19) United States
(12) Patent Application Publication
(10) Pub. No.: US 2021/0196714 A1
(43) Pub. Date: Jul. 1, 2021

(54) COMPOSITIONS AND METHODS FOR TREATING DISORDERS CHARACTERIZED WITH ABERRANT RAS/MAPK SIGNALING

(71) Applicant: Duke University, Durham, NC (US)

(72) Inventors: Nicholas KATSANIS, Durham, NC (US); I-Chun TSAI, Durham, NC (US); Spencer MCKINSTRY, Durham, NC (US); Erica DAVIS, Durham, NC (US)

(21) Appl. No.: 17/056,475

(22) PCT Filed: Jun. 18, 2019
(86) PCT No.: PCT/US2019/037763

§ 371 Date: Nov. 18, 2020

Related U.S. Application Data
(60) Provisional application No. 62/686,148, filed on Jun. 18, 2018.

(51) Int. Cl. A61K 31/506

(52) U.S. Cl. CPC A61K 31/506

(57) ABSTRACT

This invention relates generally to methods for treating, preventing and/or ameliorating symptoms of disorders characterized with aberrant RAS/MAPK signaling (e.g., Kabuki Syndrome). This invention further relates to methods and compositions for treating such disorders with pharmaceutical compositions capable of one or more of inhibiting aberrant RAS/MAPK signaling, inhibiting MEK hyperactivation related to RAS/MAPK signaling, inhibiting aberrant kmt2d, kmd6a, RAP1A, and/or RAP1B activity and/or expression related to RAS/MAPK signaling, preventing diminished RAF1 inhibition of RAP1A or RAP1B activity within RAS/C MAPK signaling, preventing or ameliorating symptoms of disorders characterized with aberrant RAS/MAPK signaling.

### PRIORITY

This application claims priority to U.S. Provisional Application No. 62/686,148, filed Jun. 18, 2018, the entire contents of which are incorporated herein by reference.

### FIELD OF THE INVENTION

This invention relates to methods for treating, preventing and/or ameliorating symptoms of disorders characterized with aberrant RAS/MAPK signaling (e.g., Kabuki Syndrome). This invention further relates to methods and compositions for treating such disorders with pharmaceutical compositions capable of one or more of inhibiting aberrant RAS/MAPK signaling, inhibiting MEK hyperactivation related to RAS/MAPK signaling, inhibiting aberrant kmt2d, kmd6a, RAP1A, and/or RAP1B activity and/or expression related to RAS/MAPK signaling, preventing diminished RAF1 inhibition of RAP1A or RAP1B activity within RAS/MAPK signaling, preventing or ameliorating symptoms of disorders characterized with aberrant RAS/MAPK signaling.

### BACKGROUND OF THE INVENTION

Kabuki syndrome (KS) is a developmental disorder characterized by a distinctive set of facial features, short stature, intellectual disability, dermatoglyphic abnormalities, and internal malformations of the cardiac, renal, gastrointestinal, and/or skeletal systems (see, Banka, S. et al. Eur J Hum Genet 20, 381-8 (2012); Bogershausen, N. & Wollnik, B. Clin Genet 83, 201-11 (2013); Kuroki, Y., et al. J Pediatr 99, 570-3 (1981); Niikawa, N., et al. J Pediatr 99, 565-9 (1981)). The global prevalence has been estimated at 1:32,000 births (see, Adam, M. P. & Hudgins, L. Clin Genet 67, 209-19 (2005)). Current treatment options for KS do not exist, with clinical care limited to the management of individual symptoms (see, Dentici, M. L. et al. Archives of Disease in Childhood 100, 158-164 (2015); Schrander-Stumpel, C. T. R. M. et al. American Journal of Medical Genetics Part A 132A, 234-243 (2005)). The lack of a KS-specific treatment has motivated research into the genetic and pathomechanistic bases of the disorder, although the rarity of this syndrome continues to pose commercial and regulatory challenges in the pursuit of novel therapeutic approaches.

Accordingly, there is an urgent need to develop novel therapies for treating KS.

The present invention addresses this need.

### SUMMARY

Kabuki Syndrome (KS) is a rare disorder characterized by distinctive facial features, short stature, skeletal abnormalities, and neurodevelopmental deficits. Previously, it has been shown that loss of function of RAP1A, a RAF1 regulator, can activate the RAS/MAPK pathway and cause KS, an observation recapitulated in other genetic models of the disorder. These data suggested that suppression of this signaling cascade might be of therapeutic benefit for some features of KS. To pursue this possibility, experiments conducted during the course of developing embodiments for the present invention performed a focused small molecule screen of a series of RAS/MAPK pathway inhibitors, where the ability of such RAS/MAPK pathway inhibitors to rescue disease-relevant phenotypes in a zebrafish model of the most common KS locus, kmt2d. Consistent with a pathway-driven screening paradigm, two of 27 compounds showed reproducible rescue of early developmental pathologies. Further analyses showed that one compound, desmethyl-Dabrafenib (dmDf), induced no overt pathologies in zebrafish embryos but could rescue MEK hyperactivation in vivo and, concomitantly, structural KS-relevant phenotypes in all KS zebrafish models (kmt2d, kmd6a and rap1). Mass spectrometry quantitation suggested that a 100 nM dose resulted in sub-nanomolar exposure of this inhibitor and was sufficient to rescue both mandibular and neurodevelopmental defects. Crucially, germline kmt2d mutants recapitulated the gastrulation movement defects, micrognathia and neurogenesis phenotypes of transient models; treatment with dmDf ameliorated all of them significantly. Taken together, these data reinforce a causal link between MEK hyperactivation and KS and indicate that chemical suppression of BRAF as having clinical utility for some features of this disorder.

Accordingly, the present invention provides methods for treating, preventing and/or ameliorating symptoms of disorders characterized with aberrant RAS/MAPK signaling (e.g., KS, capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, Legius syndrome, Noonan syndrome with multiple lentigines, somatic tumors) through one or more of inhibiting aberrant RAS/MAPK signaling, inhibiting MEK hyperactivation related to RAS/MAPK signaling, inhibiting aberrant kmt2d, kmd6a, RAP1A, and/or RAP1B activity and/or expression related to RAS/MAPK signaling, preventing diminished RAF1 inhibition of RAP1A or RAP1B activity within RAS/MAPK signaling, preventing or ameliorating symptoms of disorders characterized with aberrant RAS/MAPK signaling (e.g., craniofacial dysmorphisms, musculoskeletal abnormalities, cutaneous abnormalities, cardiac abnormalities, neurocognitive impairments, and immune deficits).

Such methods are not limited to use of a particular agent capable of treating, preventing the onset of and/or ameliorating symptoms of disorders characterized with aberrant RAS/MAPK signaling (e.g., KS, capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, Legius syndrome, Noonan syndrome with multiple lentigines) through one or more of inhibiting aberrant RAS/MAPK signaling, inhibiting MEK hyperactivation related to RAS/MAPK signaling, inhibiting aberrant kmt2d, kmd6a, RAP1A, and/or RAP1B activity and/or expression related to RAS/MAPK signaling, preventing diminished RAF1 inhibition of RAP1A or RAP1B activity within RAS/MAPK signaling, preventing or ameliorating symptoms of disorders characterized with aberrant RAS/MAPK signaling (e.g., craniofacial dysmorphisms, musculoskeletal abnormalities, cutaneous abnormalities, cardiac abnormalities, neurocognitive impairment, and immune deficits).

In some embodiments, the agent is a pharmaceutical composition comprising a kinase inhibitor that inhibits the activity of BRAF, MEK and/or ERK. In some embodiments, the kinase inhibitor is an ERK inhibitor comprising the following formula:

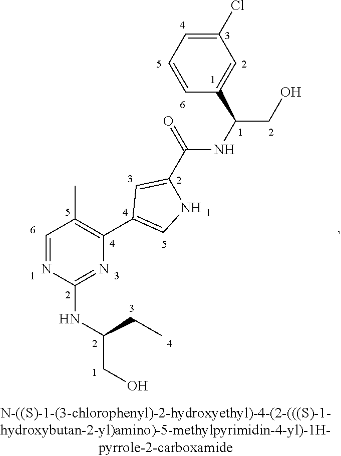 [SMILES: CC[C@@H](CO)Nc1ncc(C)c(-c2c[nH]c(C(=O)N[C@H](CO)c3cccc(Cl)c3)c2)n1]

and any derivatives, salts, and esters thereof. In some embodiments, the kinase inhibitor is an BRAF inhibitor comprising the following formula:

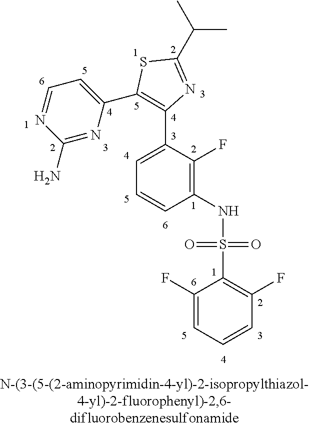 [SMILES: CC(C)c1nc(-c2cccc(NS(=O)(=O)c3c(F)cccc3F)c2F)c(-c2ccnc(N)n2)s1]

(desmethyl-Dabrafenib (dmDf)), and any derivatives, salts, and esters thereof.

In certain embodiments, the present invention provides a method for inhibiting aberrant RAS/MAPK signaling in a subject suffering from or at risk of suffering from a disorder characterized with aberrant RAS/MAPK signaling (e.g., KS, capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, Legius syndrome, Noonan syndrome with multiple lentigines) comprising, consisting of, or consisting essentially of administering to the subject a therapeutically effective amount of a pharmaceutical composition comprising a kinase inhibitor that inhibits the activity of BRAF, MEK and/or ERK. In some embodiments, the kinase inhibitor is an ERK inhibitor (e.g., N-((S)-1-(3-chlorophenyl)-2-hydroxyethyl)-4-(2-(((S)-1-hydroxybutan-2-yl)amino)-5-methylpyrimidin-4-yl)-1H-pyrrole-2-carboxamide and any derivatives, salts, and esters thereof) or a BRAF inhibitor (e.g., dmDf, and any derivatives, salts, and esters thereof).

In certain embodiments, the present invention provides a method for inhibiting MEK hyperactivation related to RAS/MAPK signaling in a subject suffering from or at risk of suffering from a disorder characterized with aberrant RAS/MAPK signaling (e.g., KS, capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, Legius syndrome, Noonan syndrome with multiple lentigines) comprising, consisting of, or consisting essentially of administering to the subject a therapeutically effective amount of a pharmaceutical composition comprising a kinase inhibitor that inhibits the activity of BRAF, MEK and/or ERK. In some embodiments, the kinase inhibitor is an ERK inhibitor (e.g., N-((S)-1-(3-chlorophenyl)-2-hydroxyethyl)-4-(2-(((S)-1-hydroxybutan-2-yl)amino)-5-methylpyrimidin-4-yl)-1H-pyrrole-2-carboxamide and any derivatives, salts, and esters thereof) or a BRAF inhibitor (e.g., dmDf, and any derivatives, salts, and esters thereof).

In certain embodiments, the present invention provides a method for inhibiting aberrant kmt2d, kmd6a, RAP1A, and/or RAP1B activity and/or expression related to RAS/MAPK signaling in a subject suffering from or at risk of suffering from a disorder characterized with aberrant RAS/MAPK signaling (e.g., KS, capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, Legius syndrome, Noonan syndrome with multiple lentigines) comprising, consisting of, or consisting essentially of administering to the subject a therapeutically effective amount of a pharmaceutical composition comprising a kinase inhibitor that inhibits the activity of BRAF, MEK and/or ERK. In some embodiments, the kinase inhibitor is an ERK inhibitor (e.g., N-((S)-1-(3-chlorophenyl)-2-hydroxyethyl)-4-(2-(((S)-1-hydroxybutan-2-yl)amino)-5-methylpyrimidin-4-yl)-1H-pyrrole-2-carboxamide and any derivatives, salts, and esters thereof) or a BRAF inhibitor (e.g., dmDf, and any derivatives, salts, and esters thereof).

In certain embodiments, the present invention provides a method for preventing diminished RAF1 inhibition of RAP1A or RAP1B activity within RAS/MAPK signaling in a subject suffering from or at risk of suffering from a disorder characterized with aberrant RAS/MAPK signaling (e.g., KS, capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, Legius syndrome, Noonan syndrome with multiple lentigines) comprising, consisting of, or consisting essentially of administering to the subject a therapeutically effective amount of a pharmaceutical composition comprising a kinase inhibitor that inhibits the activity of BRAF, MEK and/or ERK. In some embodiments, the kinase inhibitor is an ERK inhibitor (e.g., N-((S)-1-(3-chlorophenyl)-2-hydroxyethyl)-4-(2-(((S)-1-hydroxybutan-2-yl)amino)-5-methylpyrimidin-4-yl)-1H-pyrrole-2-carboxamide and any derivatives, salts, and esters thereof) or a BRAF inhibitor (e.g., dmDf, and any derivatives, salts, and esters thereof).

In certain embodiments, the present invention provides a method for preventing or ameliorating symptoms of disorders characterized with aberrant RAS/MAPK signaling (e.g., craniofacial dysmorphisms, musculoskeletal abnormalities, cutaneous abnormalities, cardiac abnormalities, neurocognitive impairment, immune deficits) in a subject suffering from or at risk of suffering from a disorder characterized with aberrant RAS/MAPK signaling (e.g., KS, capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, Legius syndrome, Noonan syndrome with multiple lentigines) comprising, consisting of, or consisting essentially of administering to the subject a therapeutically effective amount of a pharmaceutical composition comprising a kinase inhibitor that inhibits the activity of BRAF, MEK and/or ERK. In some embodiments, the kinase inhibitor is an ERK inhibitor (e.g., N-((S)-1-(3-chlorophenyl)-2-hydroxyethyl)-4-(2-(((S)-1-hydroxybutan-2-yl)amino)-5-methylpyrimidin-4-yl)-1H-pyrrole-2-carboxamide and any derivatives, salts, and esters thereof) or a BRAF inhibitor (e.g., dmDf, and any derivatives, salts, and esters thereof).

In certain embodiments, the present invention provides a method for preventing or ameliorating symptoms of KS in a subject suffering from or at risk of suffering from KS comprising, consisting of, or consisting essentially of administering to the subject a therapeutically effective amount of a pharmaceutical composition comprising a kinase inhibitor that inhibits the activity of BRAF, MEK and/or ERK. In some embodiments, the kinase inhibitor is an ERK inhibitor (e.g., N-((S)-1-(3-chlorophenyl)-2-hydroxyethyl)-4-(2-(((S)-1-hydroxybutan-2-yl)amino)-5-methylpyrimidin-4-yl)-1H-pyrrole-2-carboxamide and any derivatives, salts, and esters thereof) or a BRAF inhibitor (e.g., dmDf, and any derivatives, salts, and esters thereof).

Such methods are not limited to a particular type of subject. In some embodiments, the subject is a mammal. In some embodiments, the subject is a human subject. In some embodiments, the subject is a human subject.

In certain embodiments, the present invention is also directed to methods of screening agents for treating, preventing and/or ameliorating symptoms of disorders characterized with aberrant RAS/MAPK signaling (e.g., KS, capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, Legius syndrome, Noonan syndrome with multiple lentigines) in a mammal. In some embodiments, such methods comprise administering to a patient suffering from, for example, such a disorder a candidate agent, and comparing the result of such administration to an established norm in terms of ability to inhibit aberrant RAS/MAPK signaling, inhibit MEK hyperactivation related to RAS/MAPK signaling, inhibit aberrant kmt2d, kmd6a, RAP1A, and/or RAP1B activity and/or expression related to RAS/MAPK signaling, prevent diminished RAF1 inhibition of RAP1A or RAP1B activity within RAS/MAPK signaling, prevent or ameliorate symptoms of disorders characterized with aberrant RAS/MAPK signaling (e.g., craniofacial dysmorphisms, musculoskeletal abnormalities, cutaneous abnormalities, cardiac abnormalities, neurocognitive impairment, immune deficits).

### BRIEF DESCRIPTION OF THE DRAWINGS

FIG. 1: In vivo screen using BRAF/MEK/ERK inhibitors. (a) Flow chart of the experimental design. We started the screening with 29 compounds (27 active BRAF/MEK/ERK inhibitors, and 2 negative controls). 26 compounds passed the solubility test and 24 of those passed the toxicity test. For the primary screen, 24 compounds were assessed for their ability to rescue the convergent-extension (CE) phenotype of zebrafish kmt2d morphants, followed by rescue tests of additional phenotypes (jaw and brain) within the kmt2d morphant, or CE of additional genetic KS models. (b) Toxicity results. Twenty embryos at the 8-cell stage were incubated in egg water containing 100 nM, 1 μM, and 10 μM of the 26 compounds. After 24 hours, larvae were scored for toxicity. Black bar: death; grey bar: abnormal morphology; white bar: nontoxic. (c) Embryos were injected with kmt2d-MO at the 2-4 cell stage and soaked in egg water containing 100 nM of each compound at the 8-cell stage. After 24 hours, larvae were scored for CE. Depletion of kmt2d resulted in mild (Class I, orange) and severe (Class II, blue) CE deficiency during gastrulation. The efficacy of each compound was determined by a chi-square test in which Class I and Class II were considered together. Note that the compounds were tested in two groups (compounds 1-19 and compounds 20-29), each with its own set of controls. Compound 29, an inactive Braf inhibitor, served as a negative control. *p<0.05 and **p<0.01 for compounds that improved the phenotype compared to kmt2d-MO alone.

FIG. 2: Chemical structure of compounds that rescued the CE and jaw defects in zebrafish KS models. (a) Compound 2, an ERK inhibitor. (b) Compound 8, the BRAF inhibitor desmethyl-Dabrafenib.

FIG. 3: Compounds 2 and 8 ameliorate CE and jaw defects caused by loss of kmt2d. (a) Compounds 2 and 8, but not 28 (negative control), rescue CE defects of kmt2d morphants. (b) Alcian blue staining of jaw. Depletion of kmt2d leads to a change in jaw layout, reducing the distance between Meckel's (MK) cartilage and ceratohyal (CH) cartilage (red double arrow). Treatment with compound 2 or 8, but not 28, ameliorates this defect in kmt2d morphants. (c) Quantitative measurement of the distance between MK and CH cartilages (n>25). *p<0.05; **p<0.01; ***p<0.001. Error bars show SEM (standard error of the mean).

FIG. 4: Compound 8 ameliorates the CE and jaw defects caused by loss of rap/and kdm6a. (a) gRNA targeting both zebrafish rap1a and rap1b were injected into embryos at the 1-cell stage with Cas9 protein. Larvae were incubated with compound 8 (100 nM) at the 8-cell stage and scored for micrognathia. 5 dpf larvae were stained with Alcian blue to assess the layout of the jaw cartilage. In comparison to control embryos (top), rap1-CRISPR embryos have smaller jaws. This phenotype can be ameliorated by treatment with compound 2 and 8. (b) Quantitative measurement of the distance between MK and CH cartilages. (c) Larvae were scored for CE (n>30 for each concentration). (d) 5 dpf embryos were stained with Alcian blue to assess the layout of jaw cartilage. In comparison to control embryos (top), kdm6a-MO embryos exhibit smaller jaws. This phenotype can be ameliorated by treatment with compound 8. (e) Quantitative measurement of the distance between MK and CH cartilages. *p<0.05; ***p<0.001. Error bars show SEM (standard error of the mean).

FIG. 5: Successful delivery of small molecules into zebrafish embryos. (a) Experimental design and sample preparation for the mass spectrometry (MS) analysis of compound concentration. (b) Quantitative MS analysis results of compound exposure in embryos. The right-most two columns give the concentrations of each compound detected by MS in zebrafish, quantifying in vivo exposures resulting from egg water compound doses of 100 nM and 1 μM.

FIG. 6: The efficacy of Dabrafenib, a BRAF inhibitor, to rescue KS defects. kmt2d morphants were treated with serially increasing concentrations of Dabrafenib. The embryos were scored for CE phenotype as described earlier (n>30 for each concentration).

FIG. 7: dmDf attenuates hyperactive MEK signaling in vivo. (a) Western blot analysis for the level of pMEK1/2 from the head lysate of 5 dpf zebrafish embryos. Depletion of kmt2d (lane 4) elevates the abundance of pMEK1/2 in comparison to control embryos (lane 1). Treatment with dmDf (100 nM and 500 nM, in lanes 5 and 6, respectively) attenuates hyperactivation of MEK1/2. Full-length blots are presented in FIG. 14. (b) Summary of the relative levels of pMEK1/2 from three independent experiments. *p<0.05 (n=3). (c) kmt2d morphants were treated with serially increasing concentrations of dmDf. The embryos were scored for CE phenotypes as described earlier (n>25 for each concentration. (d) The distance between MK and CH cartilages was measured from the same embryos as in (c). *p<0.05; **p<0.01. Error bars show SEM (standard error of the mean).

FIG. 8: Validation of genome editing efficiency in kmt2d-CRISPR FOs. (a) Schematic representation of the zebrafish kmt2d transcript. Blue boxes: exons; red box: gRNA targeted site; red arrowheads: primers for amplifying the potential mutation region. (b) Polyacrylamide gel electrophoresis (15% PAGE) of 2 control and 10 kmt2d-CRISPR F0 embryos shows the presence of heteroduplexes, indicating targeting events in CRISPR FOs. The PCR products of the embryos annotated with an asterisk (*) were then cloned into pCR4 for Sanger sequencing. (c) Representative Sanger sequencing results for an un-injected control embryo and 12 randomly selected clones of F0 embryos (F0-01 and F0-02) injected with kmt2d gRNA/Cas9 show insertion and or deletion events in the target region. The protospacer adjacent motif (PAM) sequence for gRNA is shown in blue box. Green box: insertion; red box: deletion; and yellow box: change.

FIG. 9: Treatment with dmDf rescues the developmental defects of kmt2d-CRISPR F0 mutants. (a) Similar to kmt2d-MO, kmt2d-CRISPR FOs also exhibit CE defects, and treatment with dmDf ameliorates the CE defects in kmt2d-CRISPR F0 embryos. (b) 5 dpf embryos were stained with Alcian blue to visualize jaw layout. kmt2d-CRISPR FOs exhibit the same change in jaw layout as kmt2d-MOinjected embryos, with a reduced distance between MK and CH cartilages. Treatment with dmDf ameliorates the jaw defects seen in kmt2d-CRISPR F0 embryos. (c) Quantitative measurement of the distance between MK and CH cartilages. ***: p<0.001. Error bars show SEM (standard error of the mean).

FIG. 10: dmDf ameliorates cell proliferation defects in the brain caused by loss of kmt2d or rap1. (a) Depletion of kmtd2 and kdm6a through morpholino injection leads to a significant reduction in the number of proliferating cells in the brain, visualized with immunostaining for phospho-histone H3 (pHH3) positive cells, in 2 dpf embryos. Treatment with dmDf significantly increases pHH3+ proliferating cells. (b) kmt2d-CRISPR and rap1-CRISPR embryos also exhibit a significant reduction in pHH3+ cells in the brain that can be rescued by treatment with dmDf. *p<0.05; **p<0.01; ***p<0.001. Error bars show SEM (standard error of the mean).

FIG. 11: Mutation of stable kmt2d-CRISPR mutants. kmt2d mutants carry a 10 bp deletion in exon 6 (red DNA sequence). This deletion results in a frameshift and premature termination that is predicted to encode maximally a 191-residue peptide (instead of a full length 4967-amino acid long protein).

FIG. 12: Treatment with dmDf rescues the defects of stable kmt2d mutant. kmt2d+/− adults were in-crossed and the embryos were then collected at 1 dpf, 2 dpf and 5 dpf to assess dmDf efficacy. (a) dmDf ameliorates significantly the CE defect in kmt2d+/−×kmt2d+/− progeny. (b) Treatment with dmDf increases significantly the brain cell proliferation of kmt2d+/−×kmt2d+/− progeny. (c) administration of dmDf appears to ameliorate the differentiation delay in kmt2d+/−×kmt2d+/− progeny. (d) dmDf rescues significantly the jaw defect in kmt2d+/−×kmt2d+/− progeny. *p<0.05; **p<0.01; ***p<0.001. Error bars show SEM (standard error of the mean).

FIG. 13: Ras/MAPK signaling pathway. Schematic representation of the Ras/MAPK pathway.

FIG. 14: Full-length blots of FIG. 7a. Western blot analysis for the level of pMEK1/2 (left blot) and MEK1/2 (right blot) from the head lysate of 5 dpf zebrafish embryos.

### DEFINITIONS

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. Embodiments recited as “including,” “comprising/* or “having” certain elements are also contemplated as “consisting essentially of” and “consisting of” those certain elements.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by one of ordinary skill in the art to which this disclosure belongs.

### DETAILED DESCRIPTION

Mutations in lysine (K)-specific methyltransferase 2D (KMT2D, also known as MLL2) (see, Ng, S. B. et al. Nat Genet 42, 790-3 (2010); Li, Y. et al. Hum Genet 130, 715-24 (2011); Lu, J., Mo, G., Ling, Y. & Ji, L. Mol Med Rep 14, 3641-5 (2016); Miyake, N. et al. Am J Med Genet A 161a, 2234-43 (2013)) and lysine (K)-specific demethylase 6 A (KDM6A) (see, Miyake, N. et al. Am J Med Genet A 161a, 2234-43 (2013); Miyake, N. et al. Hum Mutat 34, 108-10 (2013); Lederer, D. et al. Am J Hum Genet 90, 119-24 (2012)) are mutated in ˜75% and 5% of KS cases, respectively. Subsequent to these discoveries, it was reported that mutations in the genes coding for two RAS-related proteins, RAP1A and RAP1B, can also cause KS and the phenotypically-overlapping Hadziselimovic syndrome. Grounded on these observations and the known role of RAP1 in the regulation of RAS/MAPK signaling (see, Bos, J. L., de Rooij, J. & Reedquist, K. A. Nat Rev Mol Cell Biol 2, 369-77 (2001)), it was shown in zebrafish embryos that dysfunction of any of kmt2d, kdm6a, rap1a, or rap1b yields anatomical developmental defects relevant to the KS phenotype through aberrant hyperactivation of MEK within the RAS pathway (see, Bogershausen, N. et al. J Clin Invest 125, 3585-99 (2015)). During gastrulation, these phenotypes manifested as convergence and extension (CE) defects; later in development, cell-cell intercalation pathologies were observed that likely drive mandibular formation defects and, ultimately, micrognathia (see, Bogershausen, N. et al. J Clin Invest 125, 3585-99 (2015)).

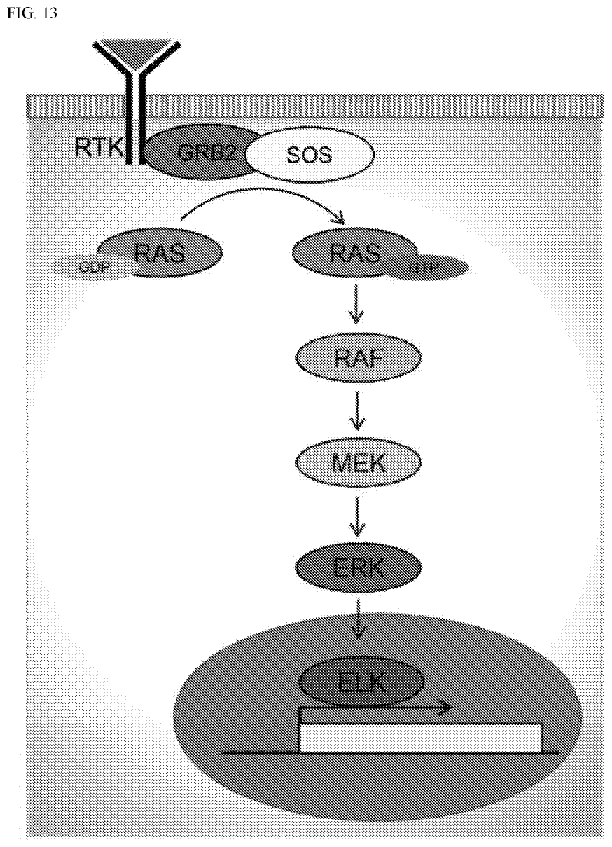

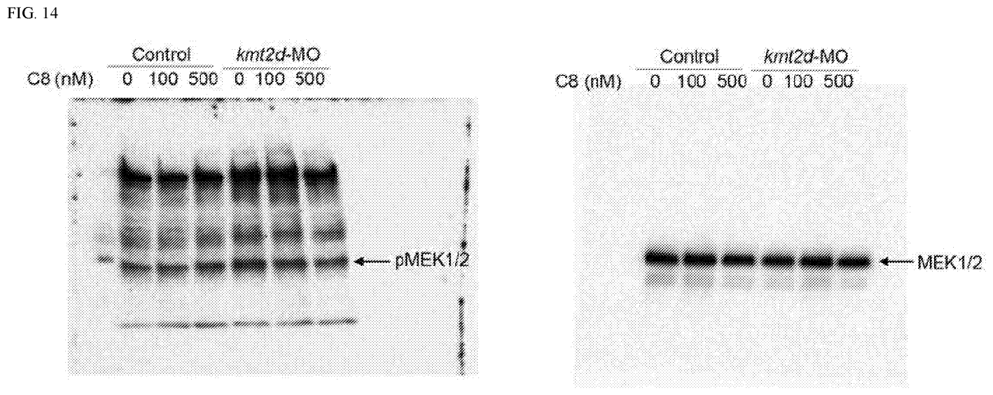

The RAS/MAPK pathway begins with RAS activation, which promotes the activation of RAF protein kinases, including ARAF, BRAF, and/or RAF1 (see, FIG. 13). RAF kinases phosphorylate and activate MEK1 and/or MEK2, which in turn phosphorylate and activate ERK1 and/or ERK2. ERK1/2 is the ultimate effector; its substrates include nuclear components, transcription factors, membrane proteins, and protein kinases that control a multitude of processes such as cell cycle progression, differentiation, and growth (see, Yoon, S. & Seger, R. Growth Factors 24, 21-44 (2006)).

In humans, the small GTPases RAP1A and RAP1B regulate RAS/MAPK signaling. In some contexts, these proteins act to inhibit the phosphorylation of RAF1, and are thus antagonists of MAPK signal propagation (see, Hu, C. D. et al. J Biol Chem 272, 11702-5 (1997)). In other contexts, RAP1A and RAP1B activate BRAF and thus agonize MAPK signaling (see, Vossler, M. R. et al. Cell 89, 73-82 (1997); York, R. D. et al. Nature 392, 622-6 (1998)). In KS, experiments have shown that loss of the RAF1-inhibitory activity of RAP1A or RAP1B is the likely driver of developmental pathologies, not least because we were able to rescue CE-driven phenotypes by suppressing RAF1 genetically in rap1 mutants. Moreover, experiments were able to phenocopy this rescue by downregulating MEK signaling by exposing morphant KS embryos to the small molecule tool compound PD184161, a MEK inhibitor (see, Bogershausen, N. et al. J Clin Invest 125, 3585-99 (2015)).

Together, these results suggested that some of the features found in KS patients overlap mechanistically with the “RASopathies,” a group of disorders caused by germline mutations in genes that encode components or regulators of the RAS/MAPK pathway (see, Tidyman, W. E. & Rauen, K. A. Curr Opin Genet Dev 19, 230-6 (2009); Rauen, K. A. Annu Rev Genomics Hum Genet 14, 355-69 (2013)). Although each RASopathy is unique, they all share characteristics with KS, such as craniofacial dysmorphisms; musculoskeletal, cutaneous, and cardiac abnormalities; and neurocognitive impairment (see, Tidyman, W. E. & Rauen, K. A. Curr Opin Genet Dev 19, 230-6 (2009)). Importantly, this group of disorders has remained too rare to motivate robust ab initio drug discovery efforts. However, the RASopathies may benefit from a serendipitous advantage, in that persistent activation of the RAS/MAPK pathway has been reported in several cancers (see, Santarpia, L., Lippman, S. M. & El-Naggar, A. K. Expert Opin Ther Targets 16, 103-19 (2012)). Most notably, activating mutations in BRAF lead to constitutive activation and phosphorylation of MEK and ERK in the RAS-RAF-MAPK signaling cascade, which are understood to contribute significantly to malignant melanoma, thyroid and colon carcinomas, as well as other cancers (see, Davies, H. et al. Nature 417, 949-54 (2002)) As a consequence, drug discovery efforts have led to the development of clinically approved inhibitors that are now prescribed for these cancers (see, Gibney, G. T. & Zager, J. S. Expert Opin Drug Metab Toxicol 9, 893-9 (2013)).

The conceptual bridge between the development of small molecule inhibitors for somatic RAS/MAPK activating mutations and their possible utility in germline disorders of this pathway has some experimental support. For example, treatment of a Raf1 mouse model of Noonan syndrome with a MEK inhibitor ameliorated several key pathologies, including short stature, facial dysmorphologies, and cardiac defects (see, Wu, X. et al. The Journal of Clinical Investigation 121, 1009-1025 (2011)). Similarly, developmental brain abnormalities of a neurofibromatosis type 1 mouse model were extinguished by neonatal administration of a MEK/ERK pathway inhibitor (see, Wang, Y. et al. Cell 150, 816-30 (2012)). Some of these therapeutic avenues have now advanced to clinical trials in humans (see, Rauen, K. A. The RAS opathies. Annu Rev Genomics Hum Genet 14, 355-69 (2013)).

Experiments conducted during the course of developing embodiments for the present invention took advantage of the malleability and physiological relevance of zebrafish KS models to test this hypothesis by screening a focused collection of chemically diverse kinase inhibitors of BRAF, MEK, and ERK for their ability to ameliorate KS deficits. Several compounds were found that could rescue phenotypes in KS zebrafish models; however, the most promising result was desmethyl Dabrafenib (dmDf), a soluble BRAF inhibitor that rescues not only the CE defects of KS morphant and mutant zebrafish embryos, but also the craniofacial and neuroanatomical defects of kmt2d-, rap1- and kdm6a-depleted zebrafish larvae, across both transient and stable genetic models. This inhibitor therefore holds promise for treating some of the features of KS with a possible expansion of its utility to other RASopathies.

Accordingly, the present invention provides methods for treating, preventing and/or ameliorating symptoms of disorders characterized with aberrant RAS/MAPK signaling (e.g., KS, capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, Legius syndrome, Noonan syndrome with multiple lentigines, somatic tumors) through one or more of inhibiting aberrant RAS/MAPK signaling, inhibiting MEK hyperactivation related to RAS/MAPK signaling, inhibiting aberrant kmt2d, kmd6a, RAP1A, and/or RAP1B activity and/or expression related to RAS/MAPK signaling, preventing diminished RAF1 inhibition of RAP1A or RAP1B activity within RAS/MAPK signaling, preventing or ameliorating symptoms of disorders characterized with aberrant RAS/MAPK signaling (e.g., craniofacial dysmorphisms, musculoskeletal abnormalities, cutaneous abnormalities, cardiac abnormalities, neurocognitive impairment, immune deficits).

Such methods are not limited to use of a particular agent capable of treating, preventing the onset of and/or ameliorating symptoms of disorders characterized with aberrant RAS/MAPK signaling (e.g., KS, capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, Legius syndrome, Noonan syndrome with multiple lentigines, somatic tumors) through one or more of inhibiting aberrant RAS/MAPK signaling, inhibiting MEK hyperactivation related to RAS/MAPK signaling, inhibiting aberrant kmt2d, kmd6a, RAP1A, and/or RAP1B activity and/or expression related to RAS/MAPK signaling, preventing diminished RAF1 inhibition of RAP1A or RAP1B activity within RAS/MAPK signaling, preventing or ameliorating symptoms of disorders characterized with aberrant RAS/MAPK signaling (e.g., craniofacial dysmorphisms, musculoskeletal abnormalities, cutaneous abnormalities, cardiac abnormalities, neurocognitive impairment, immune deficits).

In some embodiments, the agent is a pharmaceutical composition comprising a kinase inhibitor that inhibits the activity of BRAF, MEK and/or ERK. In some embodiments, the kinase inhibitor is an ERK inhibitor comprising the following formula:

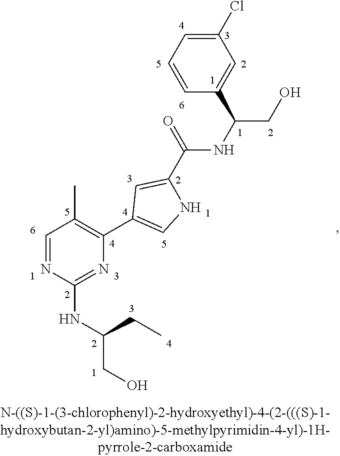 [SMILES: CC[C@@H](CO)Nc1ncc(C)c(-c2c[nH]c(C(=O)N[C@H](CO)c3cccc(Cl)c3)c2)n1]

and any derivatives, salts, and esters thereof. In some embodiments, the kinase inhibitor is an BRAF inhibitor comprising the following formula:

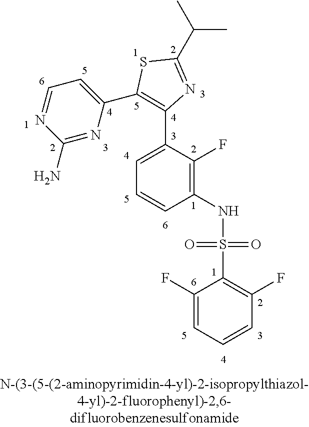 [SMILES: CC(C)c1nc(-c2cccc(NS(=O)(=O)c3c(F)cccc3F)c2F)c(-c2ccnc(N)n2)s1]

(desmethyl-Dabrafenib (dmDf)), and any derivatives, salts, and esters thereof.

In certain embodiments, the present invention provides a method for inhibiting aberrant RAS/MAPK signaling in a subject suffering from or at risk of suffering from a disorder characterized with aberrant RAS/MAPK signaling (e.g., KS, capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, Legius syndrome, Noonan syndrome with multiple lentigines) comprising, consisting of, or consisting essentially of administering to the subject a therapeutically effective amount of a pharmaceutical composition comprising a kinase inhibitor that inhibits the activity of BRAF, MEK and/or ERK. In some embodiments, the kinase inhibitor is an ERK inhibitor (e.g., N-((S)-1-(3-chlorophenyl)-2-hydroxyethyl)-4-(2-(((S)-1-hydroxybutan-2-yl)amino)-5-methylpyrimidin-4-yl)-1H-pyrrole-2-carboxamide and any derivatives, salts, and esters thereof) or a BRAF inhibitor (e.g., dmDf, and any derivatives, salts, and esters thereof).

In certain embodiments, the present invention provides a method for inhibiting MEK hyperactivation related to RAS/MAPK signaling in a subject suffering from or at risk of suffering from a disorder characterized with aberrant RAS/MAPK signaling (e.g., KS, capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, Legius syndrome, Noonan syndrome with multiple lentigines, somatic tumors) comprising, consisting of, or consisting essentially of administering to the subject a therapeutically effective amount of a pharmaceutical composition comprising a kinase inhibitor that inhibits the activity of BRAF, MEK and/or ERK. In some embodiments, the kinase inhibitor is an ERK inhibitor (e.g., N-((S)-1-(3-chlorophenyl)-2-hydroxyethyl)-4-(2-(((S)-1-hydroxybutan-2-yl)amino)-5-methylpyrimidin-4-yl)-1H-pyrrole-2-carboxamide and any derivatives, salts, and esters thereof) or a BRAF inhibitor (e.g., dmDf, and any derivatives, salts, and esters thereof).

In certain embodiments, the present invention provides a method for inhibiting aberrant kmt2d, kmd6a, RAP1A, and/or RAP1B activity and/or expression related to RAS/MAPK signaling in a subject suffering from or at risk of suffering from a disorder characterized with aberrant RAS/MAPK signaling (e.g., KS, capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, Legius syndrome, Noonan syndrome with multiple lentigines) comprising, consisting of, or consisting essentially of administering to the subject a therapeutically effective amount of a pharmaceutical composition comprising a kinase inhibitor that inhibits the activity of BRAF, MEK and/or ERK. In some embodiments, the kinase inhibitor is an ERK inhibitor (e.g., N-((S)-1-(3-chlorophenyl)-2-hydroxyethyl)-4-(2-(((S)-1-hydroxybutan-2-yl)amino)-5-methylpyrimidin-4-yl)-1H-pyrrole-2-carboxamide and any derivatives, salts, and esters thereof) or a BRAF inhibitor (e.g., dmDf, and any derivatives, salts, and esters thereof).

In certain embodiments, the present invention provides a method for preventing diminished RAF1 inhibition of RAP1A or RAP1B activity within RAS/MAPK signaling in a subject suffering from or at risk of suffering from a disorder characterized with aberrant RAS/MAPK signaling (e.g., KS, capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, Legius syndrome, Noonan syndrome with multiple lentigines) comprising, consisting of, or consisting essentially of administering to the subject a therapeutically effective amount of a pharmaceutical composition comprising a kinase inhibitor that inhibits the activity of BRAF, MEK and/or ERK. In some embodiments, the kinase inhibitor is an ERK inhibitor (e.g., N-((S)-1-(3-chlorophenyl)-2-hydroxyethyl)-4-(2-(((S)-1-hydroxybutan-2-yl)amino)-5-methylpyrimidin-4-yl)-1H-pyrrole-2-carboxamide and any derivatives, salts, and esters thereof) or a BRAF inhibitor (e.g., dmDf, and any derivatives, salts, and esters thereof).

In certain embodiments, the present invention provides a method for preventing or ameliorating symptoms of disorders characterized with aberrant RAS/MAPK signaling (e.g., craniofacial dysmorphisms, musculoskeletal abnormalities, cutaneous abnormalities, cardiac abnormalities, neurocognitive impairment, immune deficits) in a subject suffering from or at risk of suffering from a disorder characterized with aberrant RAS/MAPK signaling (e.g., KS, capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, Legius syndrome, Noonan syndrome with multiple lentigines) comprising, consisting of, or consisting essentially of administering to the subject a therapeutically effective amount of a pharmaceutical composition comprising a kinase inhibitor that inhibits the activity of BRAF, MEK and/or ERK. In some embodiments, the kinase inhibitor is an ERK inhibitor (e.g., N-((S)-1-(3-chlorophenyl)-2-hydroxyethyl)-4-(2-(((S)-1-hydroxybutan-2-yl)amino)-5-methylpyrimidin-4-yl)-1H-pyrrole-2-carboxamide and any derivatives, salts, and esters thereof) or a BRAF inhibitor (e.g., dmDf, and any derivatives, salts, and esters thereof).

In certain embodiments, the present invention provides a method for preventing or ameliorating symptoms of KS in a subject suffering from or at risk of suffering from KS comprising, consisting of, or consisting essentially of administering to the subject a therapeutically effective amount of a pharmaceutical composition comprising a kinase inhibitor that inhibits the activity of BRAF, MEK and/or ERK. In some embodiments, the kinase inhibitor is an ERK inhibitor (e.g., N-((S)-1-(3-chlorophenyl)-2-hydroxyethyl)-4-(2-(((S)-1-hydroxybutan-2-yl)amino)-5-methylpyrimidin-4-yl)-1H-pyrrole-2-carboxamide and any derivatives, salts, and esters thereof) or a BRAF inhibitor (e.g., dmDf, and any derivatives, salts, and esters thereof).

Such methods are not limited to a particular type of subject. In some embodiments, the subject is a mammal. Such mammals include humans as well as non-human mammals. Non-human mammals include, for example, companion animals such as dogs and cats, agricultural animals such live stock including cows, horses and the like, and exotic animals, such as zoo animals.

Administration of such a pharmaceutical composition comprising a kinase inhibitor that inhibits the activity of BRAF, MEK and/or ERK (e.g., N-((S)-1-(3-chlorophenyl)-2-hydroxyethyl)-4-(2-(((S)-1-hydroxybutan-2-yl)amino)-5-methylpyrimidin-4-yl)-1H-pyrrole-2-carboxamide and any derivatives, salts, and esters thereof) (dmDf, and any derivatives, salts, and esters thereof) can be by any suitable route of administration including buccal, dental, endocervical, intramuscular, inhalation, intracranial, intralymphatic, intramuscular, intraocular, intraperitoneal, intrapleural, intrathecal, intratracheal, intrauterine, intravascular, intravenous, intravesical, intranasal, ophthalmic, oral, otic, biliary perfusion, cardiac perfusion, priodontal, rectal, spinal subcutaneous, sublingual, topical, intravaginal, transermal, ureteral, or urethral. Dosage forms can be aerosol including metered aerosol, chewable bar, capsule, capsule containing coated pellets, capsule containing delayed release pellets, capsule containing extended release pellets, concentrate, cream, augmented cream, suppository cream, disc, dressing, elixer, emulsion, enema, extended release fiber, extended release film, gas, gel, metered gel, granule, delayed release granule, effervescent granule, chewing gum, implant, inhalant, injectable, injectable lipid complex, injectable liposomes, insert, extended release insert, intrauterine device, jelly, liquid, extended release liquid, lotion, augmented lotion, shampoo lotion, oil, ointment, augmented ointment, paste, pastille, pellet, powder, extended release powder, metered powder, ring, shampoo, soap solution, solution for slush, solution/drops, concentrate solution, gel forming solution/drops, sponge, spray, metered spray, suppository, suspension, suspension/drops, extended release suspension, swab, syrup, tablet, chewable tablet, tablet containing coated particles, delayed release tablet, dispersible tablet, effervescent tablet, extended release tablet, orally disintegrating tablet, tampon, tape or troche/lozenge.

Intraocular administration can include administration by injection including intravitreal injection, by eyedrops and by trans-scleral delivery.

Administration can also be by inclusion in the diet of the mammal such as in a functional food for humans or companion animals.

It is also contemplated that certain formulations containing the compositions comprising a kinase inhibitor that inhibits the activity of BRAF, MEK and/or ERK (e.g., N-((S)-1-(3-chlorophenyl)-2-hydroxyethyl)-4-(2-(((S)-1-hydroxybutan-2-yl)amino)-5-methylpyrimidin-4-yl)-1H-pyrrole-2-carboxamide and any derivatives, salts, and esters thereof) (dmDf, and any derivatives, salts, and esters thereof) are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methylcellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated such as to provide rapid, sustained, or delayed release of the active ingredients after administration to the patient by employing procedures well known in the art. The formulations can also contain substances that diminish proteolytic degradation and promote absorption such as, for example, surface-active agents.

The specific dose can be calculated according to the approximate body weight or body surface area of the patient or the volume of body space to be occupied. The dose will also depend upon the particular route of administration selected. Further refinement of the calculations necessary to determine the appropriate dosage for treatment is routinely made by those of ordinary skill in the art. Such calculations can be made without undue experimentation by one skilled in the art in light of the activity in assay preparations such as has been described elsewhere for certain compounds (see for example, Howitz et al., Nature 425:191-196, 2003 and supplementary information that accompanies the paper). Exact dosages can be determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

The present invention also provides kits comprising a kinase inhibitor that inhibits the activity of BRAF, MEK and/or ERK (e.g., N-((S)-1-(3-chlorophenyl)-2-hydroxyethyl)-4-(2-(((S)-1-hydroxybutan-2-yl)amino)-5-methylpyrimidin-4-yl)-1H-pyrrole-2-carboxamide and any derivatives, salts, and esters thereof) (dmDf, and any derivatives, salts, and esters thereof) and instructions for administering the agent to an animal (e.g., a human patient suffering from a neurodegenerative disorder (e.g., AD)). The kits may optionally contain other therapeutic agents.

### EXPERIMENTAL

The following examples are provided to demonstrate and further illustrate certain preferred embodiments of the present invention and are not to be construed as limiting the scope thereof.

### Example I. Screening of Chemical Compounds for Potential KS Treatments

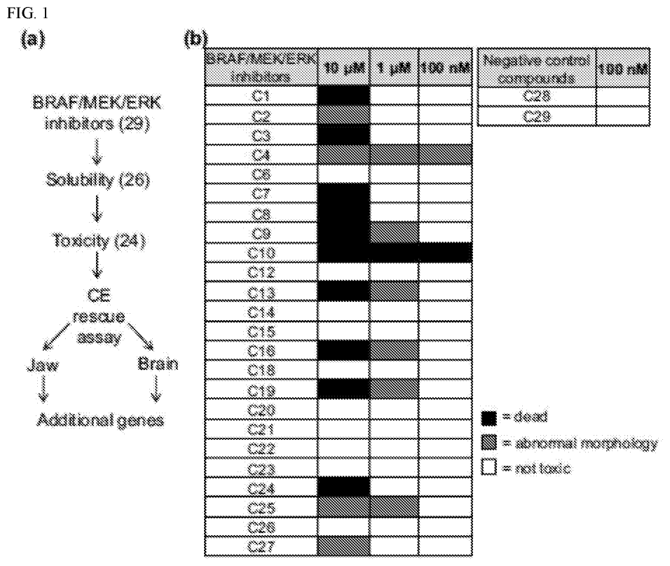

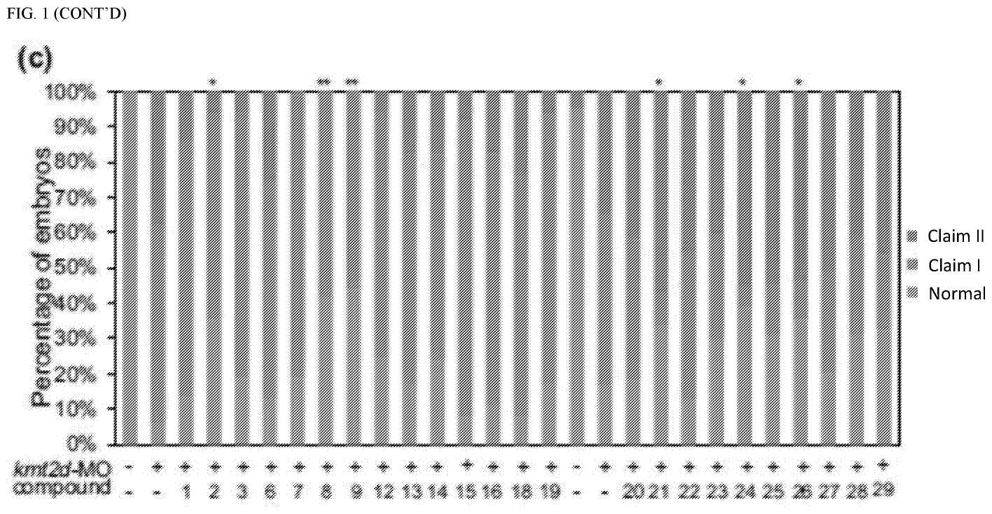

A series of 27 validated tool BRAF, MEK, and ERK antagonists were obtained, as well as two inactive BRAF inhibitor analogs. To guard against possible bias, the tool and negative control compounds were interspersed across the experiment and the zebrafish phenotyping team was blinded to the identity of each compound. Typical of small molecule storage, each of the 29 compounds was dissolved in dimethyl sulfoxide (DMSO). Therefore, such compounds were first diluted in water (100×) and its solubility tested in egg water; three compounds were insoluble and were thus removed from further testing (see, FIG. 1A). Next, each compound was diluted serially with egg water and toxicity tested by assaying the percentage of abnormal or dead embryos across three different concentrations (10 μM, 1 μM, 100 nM; FIG. 1B). It was found that 22/26 small molecules induced no appreciable pathology at a concentration of 100 nM (FIG. 1B); accordingly, that dose was selected for subsequent experiments.

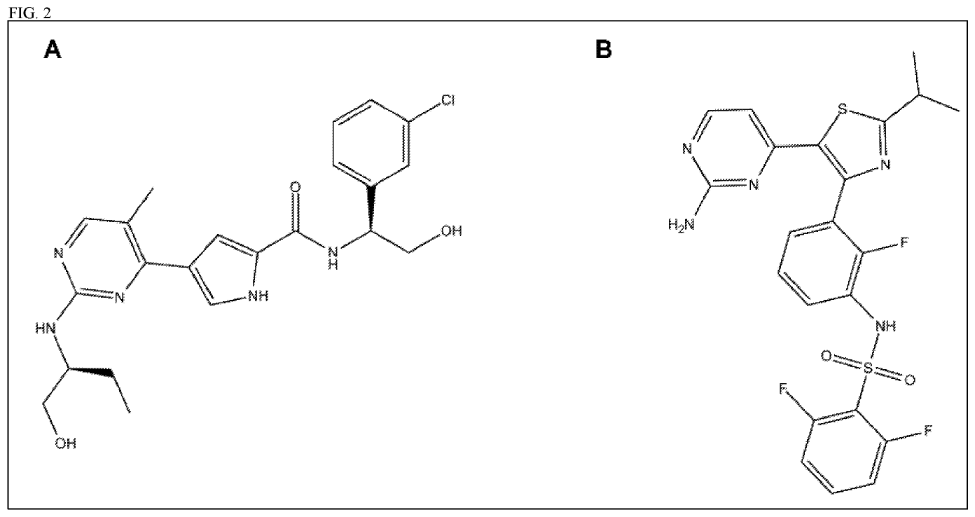

This library was screened against validated transient suppression reagent for kmt2d. Experiments injected kmt2d-morpholino (MO) into one- to two-cell-stage embryos and soaked them in egg water containing 100 nM of each compound approximately one hour after injection (eight-cell stage). Embryos were allowed to grow to the 8-10 somite stage, at which point they were scored for CE phenotypes using previously established objective qualitative criteria (normal; class I, which have grossly normal morphology, but are shorter than control-injected embryos; or class II, which are shorter and thinner than class I embryos and have poorly developed head, eye, and tail structure, with poor somitic definition and symmetry (see, Leitch, C. C. et al. Nat Genet 40, 443-8 (2008)). Compared to a vehicle control (treatment with DMSO), treatment with compounds 2, 8, 9, 21, 24, and 26 (structures of compounds 2 and 8 given in FIG. 2) decreased the proportion of embryos showing CE defects (though note that compounds 21, 24, and 26 appeared to increase the proportion falling into Class II; FIG. 1C). Reassuringly, post hoc analysis of the identity of the compounds revealed that neither of the two negative controls (compounds 28 and 29 (FIG. 1C)) had an appreciable effect.

### Example II. Compounds 2 and 8 Ameliorate CE and Jaw Defects of kmt2d, kdm6a and Rap1 Models

To identify candidates suitable for further preclinical studies, experiments were conducted that next asked (1) which of the six compounds that could rescue kmt2d MO-induced CE could be reproduced; (2) whether the compounds identified from the screen could also rescue the craniofacial anomalies seen in our KS zebrafish kmt2d morphants; and (3) whether these compounds could also rescue the defects in other KS zebrafish models.

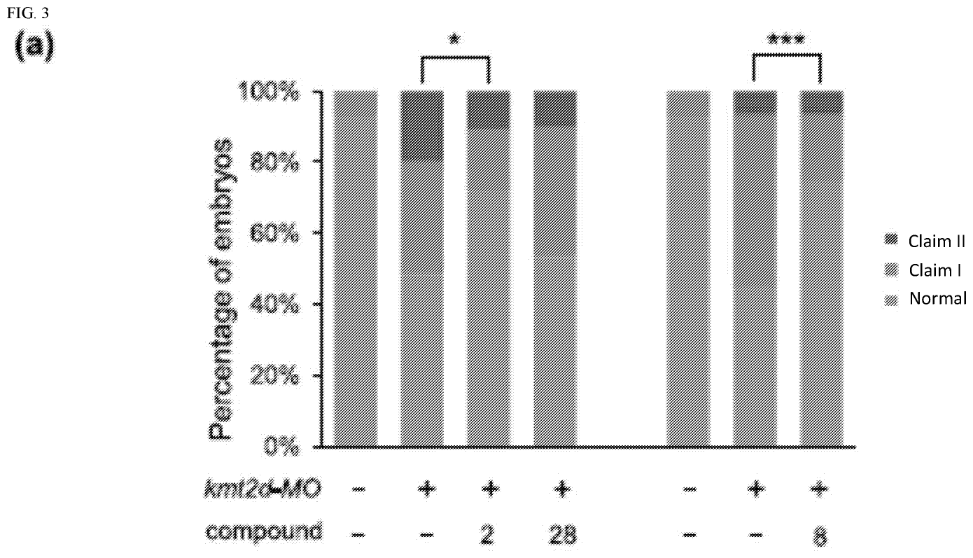

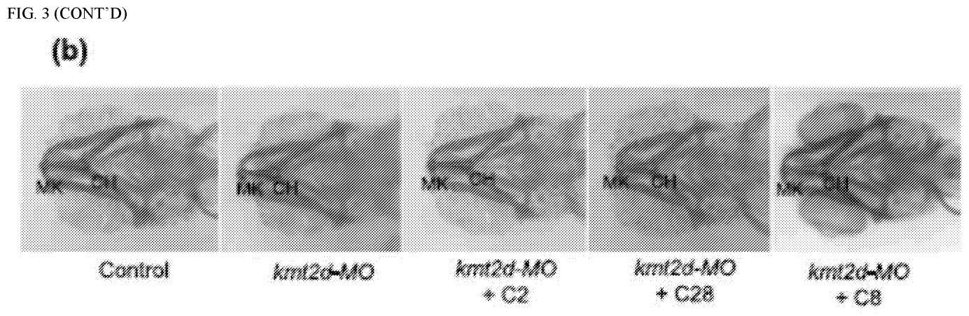

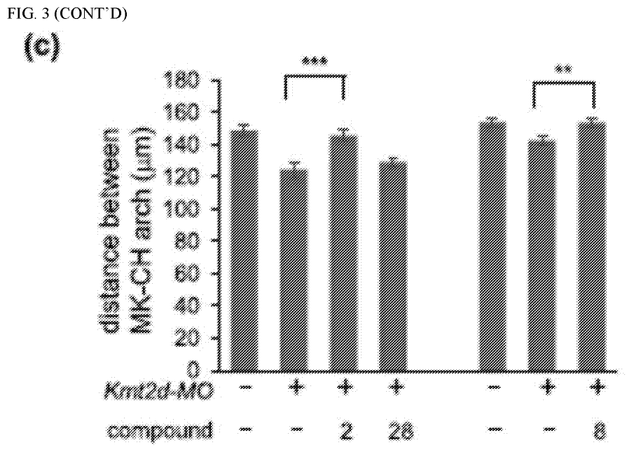

To test the first criterion, the CE rescue paradigm was repeated four times by two different investigators, each using independent zebrafish adult stocks. It was found that compounds 2 and 8 gave consistent and significant rescue of CE in kmt2d morphants across all experiments (representative data, FIG. 3). For the second criterion, larvae were raised in the presence of compound to 5 days post fertilization (dpf), fixed and stained cartilage structures with Alcian blue, and measured the distance between the Meckel's (MK) and ceratohyal (CH) cartilages, which has been shown previously to be reduced significantly in kmt2d morphants and rap1 mutants and morphants (see, Bogershausen, N. et al. et al., J Clin Invest 125, 3585-99 (2015)).

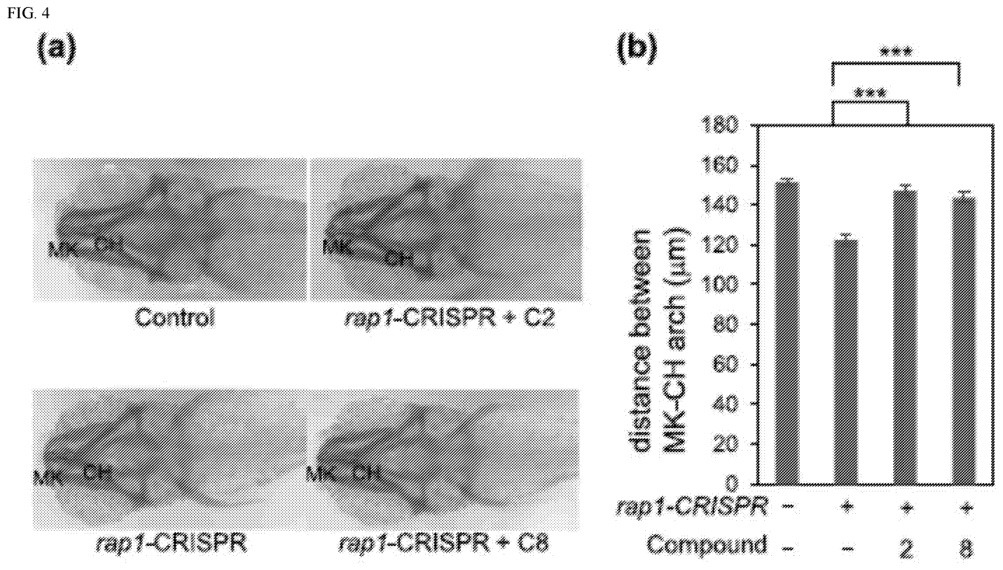

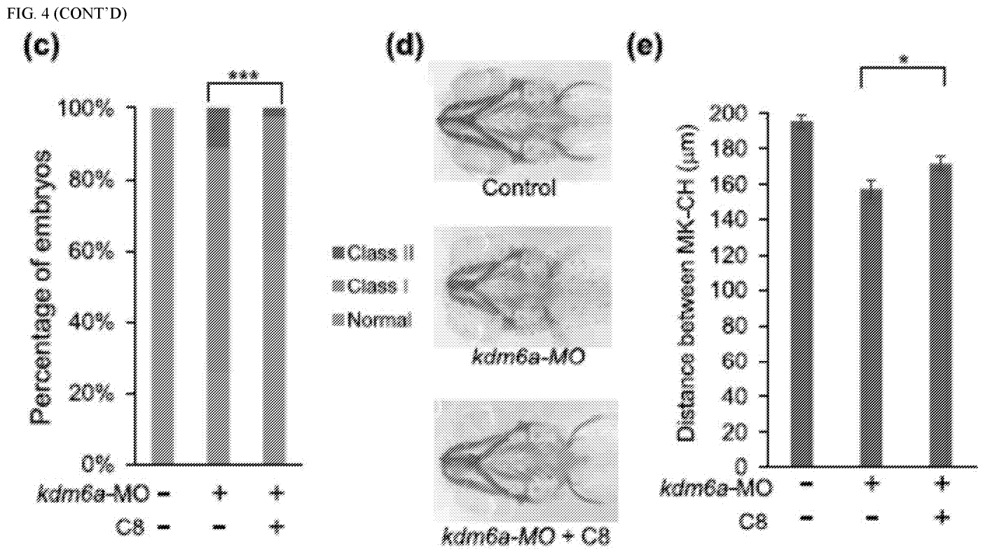

Consistent with the CE data, and with the expectation that CE defects and micrognathia are both likely driven by aberrant cell intercalation (see, Sarmah, S. et al. Plos One 5, e10367 (2010); Zoeller, J. J. et al. J Biol Chem 284, 11728-37 (2009)), it was found that treatment of kmt2d morphants with compounds 2 and 8 could rescue significantly the jaw defects as indicated by MK-CH distance (FIGS. 3B and 3C). Next, it was asked whether compounds 2 and 8 could ameliorate the jaw pathology of additional KS zebrafish models, including a kmd6a morphant (kmd6a has been refractory to CRISPR/Cas9 mutagenesis) and a rapt F0 CRISPR mutant (see, Bogershausen, N. et al. J Clin Invest 125, 3585-99 (2015)). For this purpose, experiments assayed larvae at 5 dpf using the same experimental conditions as for our kmt2d morphants. Bathing embryos in either compounds 2 or 8 (FIG. 2) restored significantly the jaw length of morphants and mutants to nearly wild type levels (FIG. 4A, 4B, 4D, 4E).

### Example III. Exposure of Compounds 2 and 8 in Zebrafish Embryos

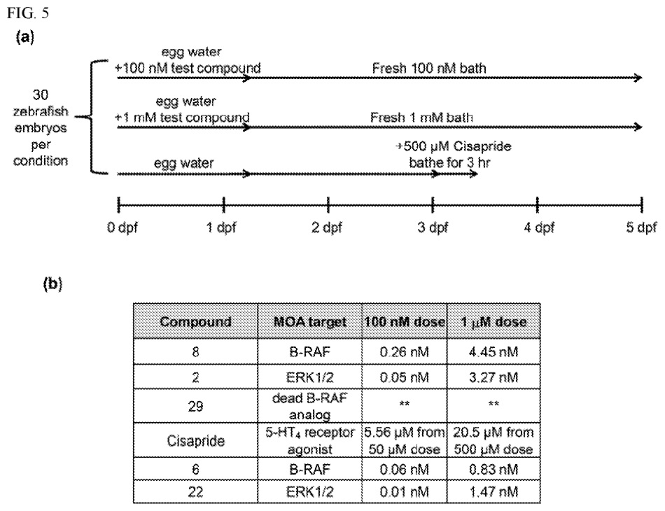

The data suggest that both compounds 2 and 8 offer proof of concept that inhibiting the MEK/ERK pathway may improve features of KS. It was therefore wondered about exposure of these molecules in the zebrafish embryos at the dosage that achieved significant rescue (100 nM). For this purpose, mass spectrometry (MS) was performed in embryos treated with each of compounds 2 and 8. Wild-type embryos were treated with 100 nM (the concentration used for the rescue experiments) or 1 μM (as a positive detection threshold given that the intracellular concentration of each compound is unknown at the beginning of the experiment). After five days of treatment, embryos were harvested and subjected to MS analysis (FIG. 5A). In addition to compounds 2 and 8, the following controls were also assayed: (1) Compound 29, an inactive BRAF inhibitor, which served as a standard for calibration; (2) Cisapride, which was included to assess the efficiency of homogenization (see, Berghmans, S. et al. J Pharmacol Toxicol Methods 58, 59-68 (2008)); and (3) Compounds 6 and 22, which are BRAF and ERK inhibitors, respectively, that did not rescue significantly the KS zebrafish models. Detectable concentrations were observed of compounds 2, 8, 6, and 22, indicating that all these compounds, including the compounds that failed to rescue KS phenotypes, can be delivered into zebrafish embryos via the bathing method (FIG. 5B).

Unmasking the identity of compounds 2 and 8 revealed the former to be an ERK inhibitor, and the latter a BRAF inhibitor, reinforcing the notion that the pathway forms a central axis in the disease pathophysiology. Compound 2 (CAS #449732-51-8) is an analog of Ulixertinib and a potent ERK2, and likely ERK1, inhibitor (see, WO 2002064586). Compound 8 is desmethyl-Dabrafenib, the desmethyl-metabolic byproduct of Dabrafenib that, in the human gut, is either excreted or reabsorbed into the bloodstream (see, Bershas, D. A. et al. Drug Metab Dispos 41, 2215-24 (2013)). Given that Dabrafenib is a marketed product (see, Ballantyne, A. D. & Garnock-Jones, K. P. Drugs 73, 1367-76 (2013); Falchook, G. S. et al. Lancet 379, 1893-901 (2012); Rutkowski, P. & Blank, C. Expert Opin Drug Saf 13, 1249-58 (2014)), it was therefore reasoned that compound 8 might likewise be able to be used in humans, whereas there are less data available for compound 2. For this reason, experiments focused on compound 8, annotated henceforth as dmDf.

Finally, the data suggest that the LC50 (lethal concentration required to kill 50% of the population) of dmDf in zebrafish falls between 1 and 10 μM (FIG. 1). This concentration is some four orders of magnitude greater than the effective in vivo exposure (0.26 nM, Supplementary FIG. 4b) of dmDf that sufficiently rescues both mandibular and neurodevelopmental defects. These findings suggest that effective low-dosing of RAS/MAPK pathway inhibitors, such as dmDf, has the potential to safely ameliorate developmental KS phenotypes in human.

### Example IV. Dabrafenib does not Rescue a Zebrafish Model of KS

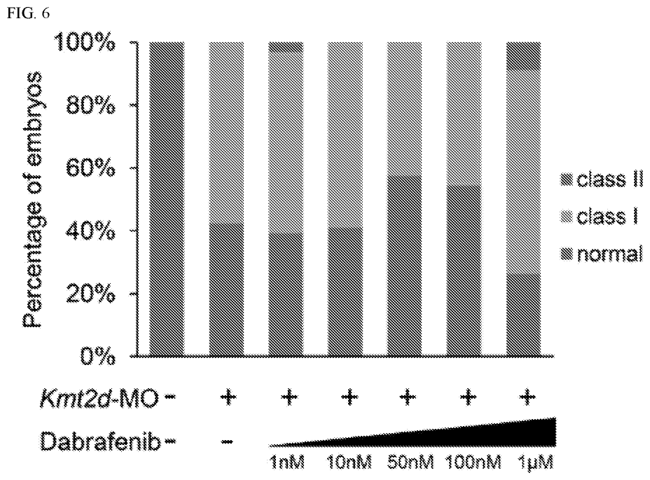

Among the numerous clinical trials involving RAS/MAPK signaling, small molecules targeting tumors that express BRAF mutations have been among the furthest to advance (see, Huang, T. et al. J Hematol Oncol 6, 30 (2013)). For example, Dabrafenib, a small molecule designed against the hyperactivated BRAF V600E mutant, has shown efficacy in adults with solid tumors, most notably malignant melanoma (see, Ballantyne, A. D. & Garnock-Jones, K. P. Drugs 73, 1367-76 (2013); Falchook, G. S. et al. Lancet 379, 1893-901 (2012); Rutkowski, P. & Blank, C. Expert Opin Drug Saf 13, 1249-58 (2014)). Given the apparent efficacy of dmDf in ameliorating KS in our zebrafish model, it was wondered whether Dabrafenib might have a similar effect, which could accelerate its administration in KS patients. kmt2d morphants were treated with five serial dilutions of Dabrafenib, (1 nM to 1 μM), and embryos scored for amelioration of CE defects at the 8-10 somite stage. Blind scoring of biological duplicates did not result in significant amelioration, with marginal improvements observed at the 50 nM and 100 nM doses (FIG. 6). This result does not preclude the potential utility of Dabrafenib in the context of human metabolism or postnatal phenotypes. However, such results indicate that, compared with Dabrafenib, dmDf potentially shows greater therapeutic promise.

### Example V. dmDf Attenuates Hyperactive MEK Signaling In Vivo

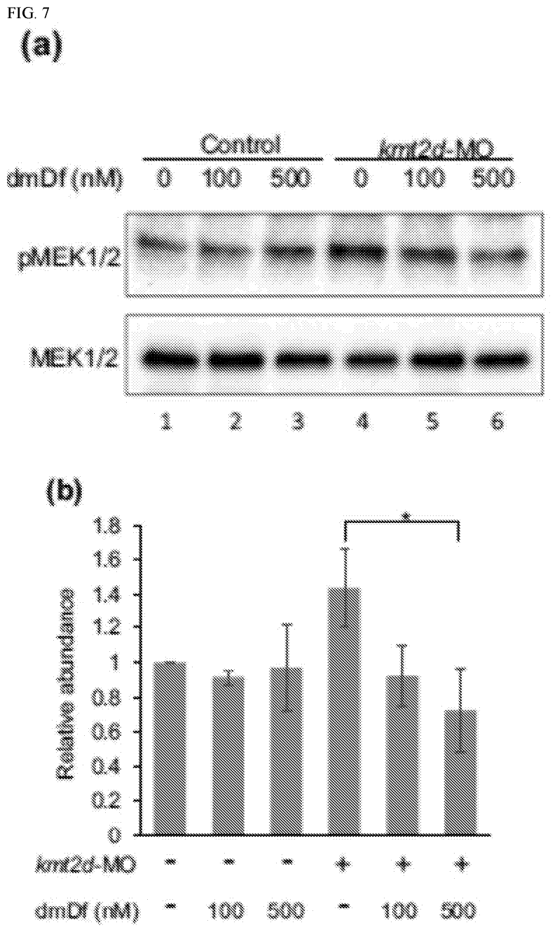

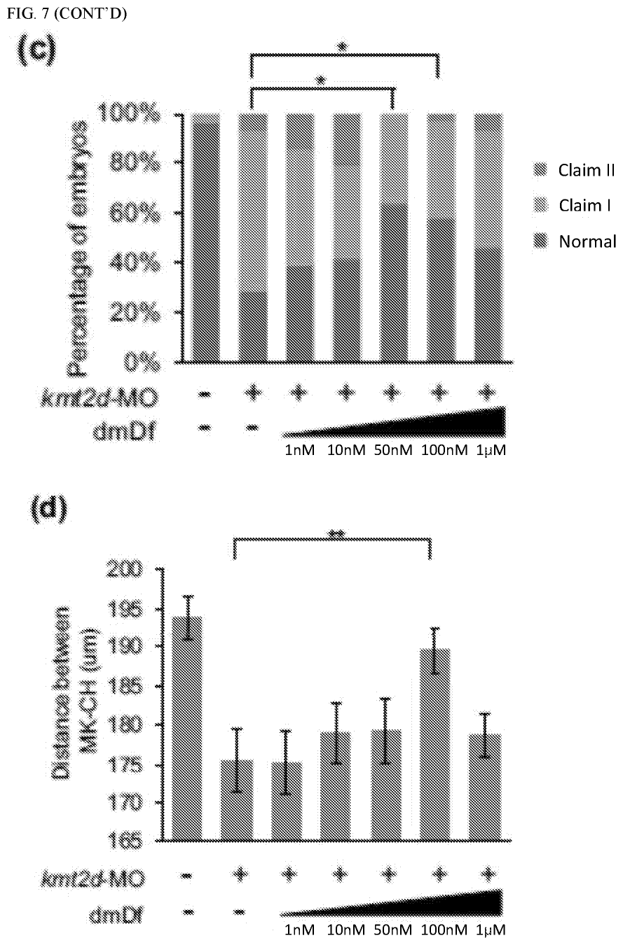

The phenotypic rescue of the zebrafish KS models by dmDf predicts a successful attenuation of hyperactive MEK signaling. To test this hypothesis directly, experiments were conducted that suppressed kmt2d, harvested and pooled protein from embryo heads (n=20/condition) and measured the abundance of phosphorylated (activated) MEK1/2 (pMEK1/2). Consistent with our morphological data, exposure of embryos to either 100 nM or 500 nM of dmDf restored the abundance of pMEK1/2 to levels indistinguishable from wild type embryos (FIGS. 7A and 7B); neither concentration had an appreciable effect on pMEK1/2 abundance in uninjected embryos, an observation consistent with the lack of toxicity of the compound at these concentrations (FIG. 1B).

Finally, we carried out an assessment on the dose response of dmDf in our KS zebrafish model. Although it is technically difficult to study molecular interactions (such as with phospho-BRAF to report the amount of free BRAF at different drug concentrations) in the zebrafish system, dose-response data can provide information about maximal efficacy and potency. Experiments treated kmt2d morphants with 1 nM to 1 μM of dmDf and the degree to which CE and jaw defects were rescued was assessed (FIGS. 7C and 7D. Significant rescue of CE defects by 50 and 100 nM of dmDf was observed, while only the 100 nM concentration of this compound was able to rescue the jaw layout (FIGS. 7C and 7D). In both features, the ability to rescue increased with drug dosage up to 100 nM. Beyond that point, higher dosages did not improve efficacy any further.

### Example VI. Potential Utility of dmDf for KS-Relevant Neuroanatomical Defects

Although the attenuation of CE and jaw defects by dmDf in our zebrafish models is encouraging, the clinical impact of the former phenotype is unclear. Experiments were conducted that therefore wondered whether we could use this compound to ameliorate phenotypes that, while still testable during zebrafish development, might be clinically relevant for young KS patients. Studies have shown that postnatal reversal of neurodevelopmental genetic insults can be efficacious in protecting against long-term neurocognitive defects if administered early in postnatal life (see, Leonard, H., Cobb, S. & Downs, J. Nat Rev Neurol (2016)), possibly because neurological development in humans continues through childhood and adolescence (see, Stiles, J. & Jernigan, T. L. Neuropsychol Rev 20, 327-48 (2010)). Under this paradigm, experiments asked whether dmDf could restore neurogenesis in genetic KS zebrafish mutants.

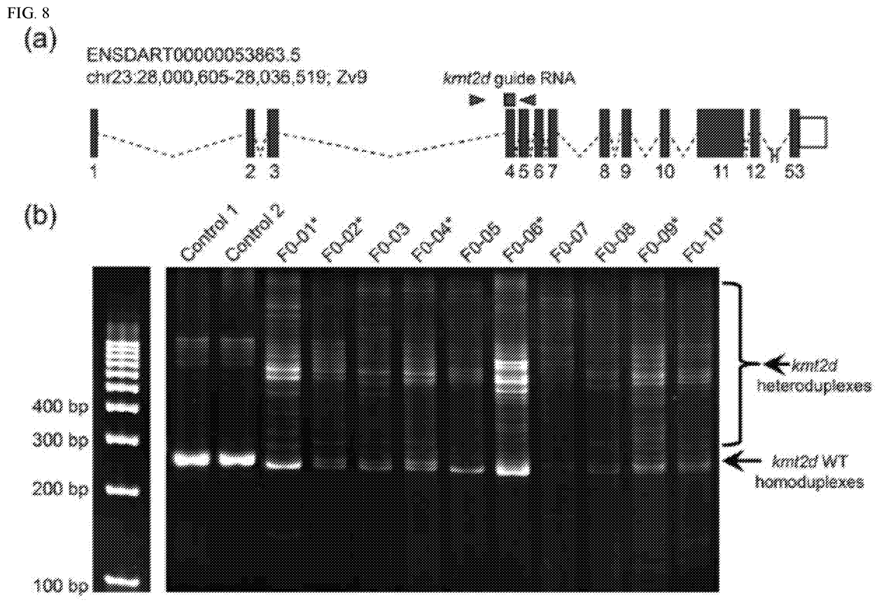

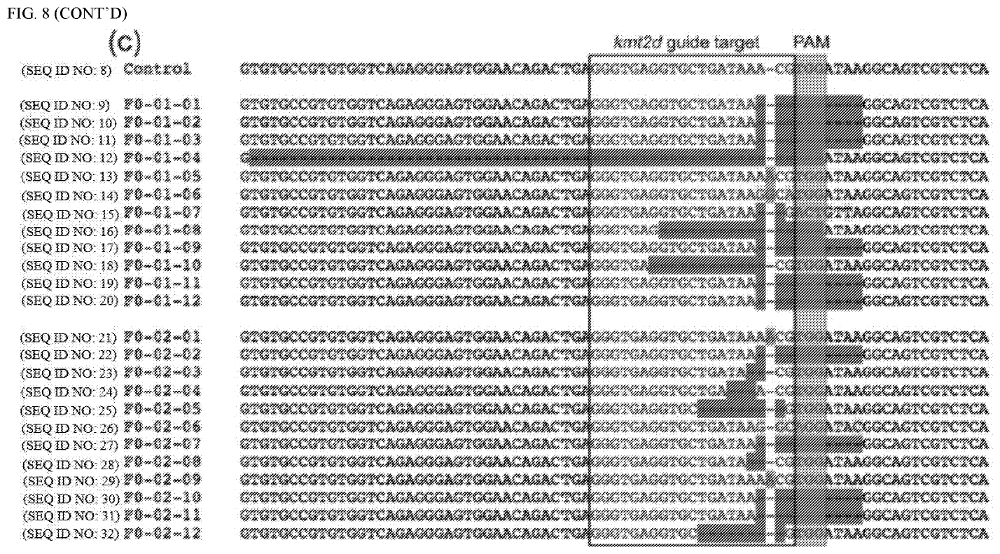

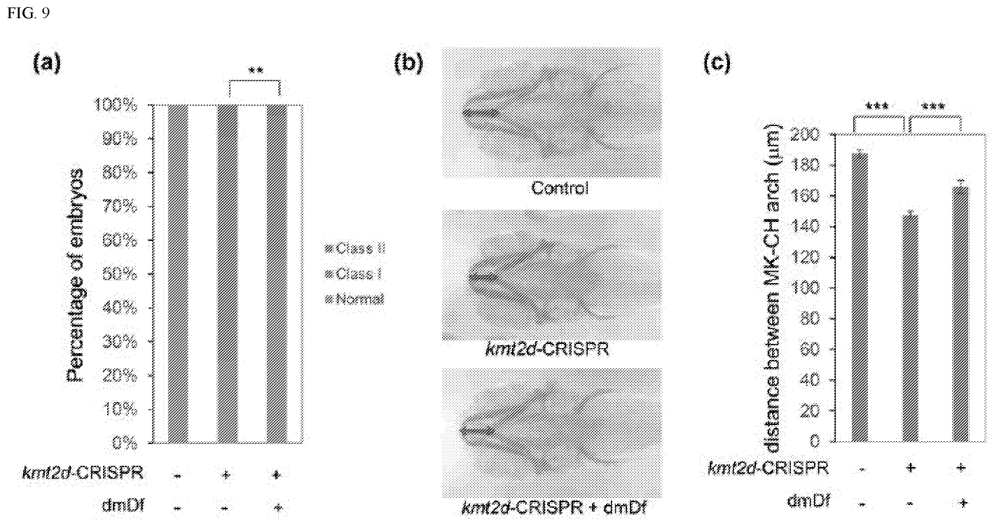

Prior and current genetic zebrafish models of KS focused on the newly discovered rap1a/rap1b genes. However, from the perspective of clinical utility, dmDf should be efficacious in kmt2d mutants, since loss of function mutations of the human ortholog account for as much as 75% of KS (see, Ng, S. B. et al. Nat Genet 42, 790-3 (2010); Li, Y. et al. Hum Genet 130, 715-24 (2011); Lu, J., Mo, G., Ling, Y. & Ji, L. Mol Med Rep 14, 3641-5 (2016); Miyake, N. et al. Am J Med Genet A 161a, 2234-43 (2013)). Therefore, experiments supplemented the testing tools by creating a kmt2d CRISPR/Cas9 mutant. A guide RNA targeting exon 4 of kmt2d induced efficient (˜99%) Cas9-mediated genome editing (FIG. 8). Similar to kmt2d morphants and rap1 CRISPR mutants, kmt2d CRISPR mosaic mutants (F0) exhibited defects in CE and jaw development; encouragingly, both phenotypes could be ameliorated significantly by administration of 100 nM of dmDf (FIGS. 9A, 9B, and 9C).

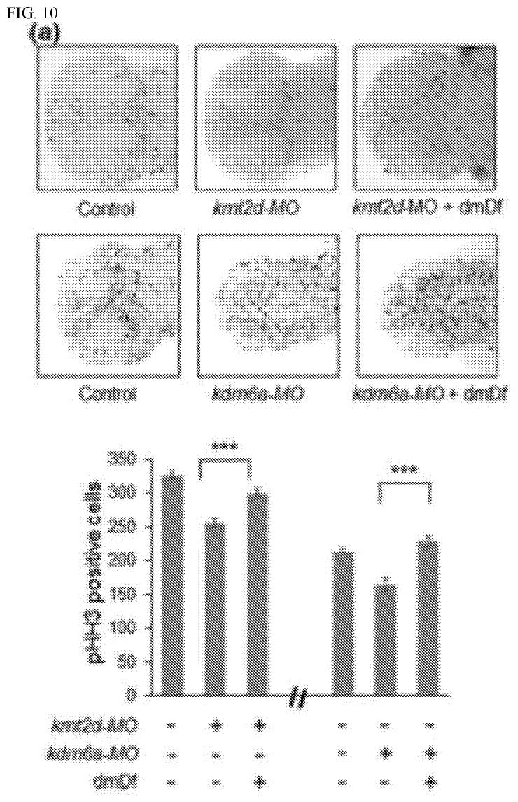

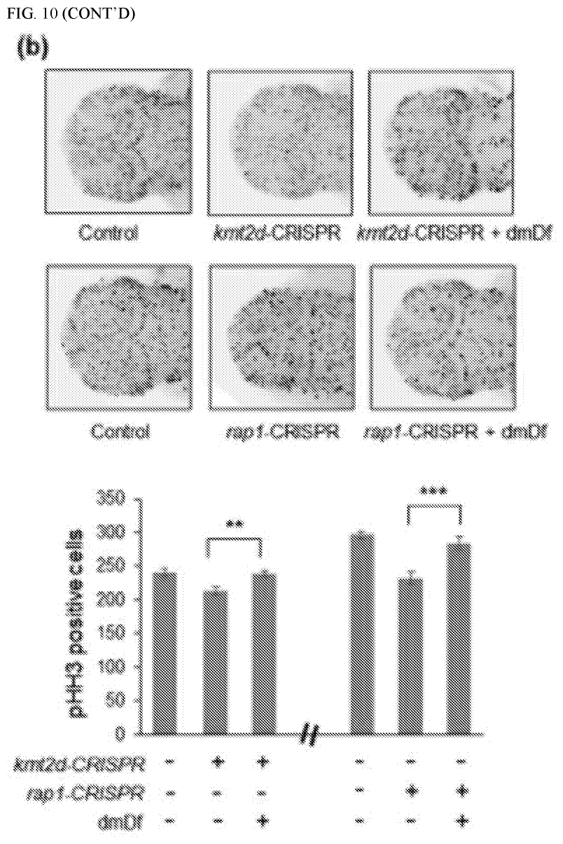

Intellectual disability represents a major phenotype in KS, with some studies reporting almost 90% of patients to be affected (see, Wessels, M. W., et al. Clin Dysmorphol 11, 95-102 (2002)). Although the mechanistic basis of this phenotype is unknown and likely driven by multiple neurodevelopmental defects, experiments were conducted that asked (1) whether the transient or stable KS zebrafish models exhibited quantitative neurodevelopmental phenotypes; and (2) whether any observed pathologies could be rescued by dmDf. Previous studies have shown that defects in proliferation in the developing zebrafish brain can be a proxy for neurodevelopmental and neurocognitive traits, especially in the context of microcephaly (see, Borck, G. et al. Genome Res 25, 609 (2015); Brooks, S. S. et al. Genetics 198, 723-33 (2014); O'Rawe, J. A. et al. Am J Hum Genet 97, 922-32 (2015); Wortmann, S. B. et al. Am J Hum Genet 96, 245-57 (2015)), a phenotype reported commonly in KS (see, Matsumoto, N. & Niikawa, N. Am J Med Genet C Semin Med Genet 117C, 57-65 (2003)). As such, experiments were conducted that quantified the total number of proliferating cells in the developing zebrafish brain by phospho-Histone H3 (pHH3) immunostaining at 2 dpf Consistent with our predictions, kmt2d and kdm6a morphants, as well as kmt2d- and rap1-mosaic mutants exhibited a significant reduction of proliferating cells in the developing brain compared to sham-injected controls (FIG. 10). In each case, treatment with 100 nM dmDf restored the total number of proliferating cells to wild type levels (FIG. 10).

### Example VII. dmDf Rescues a Stable kmt2di Mutant

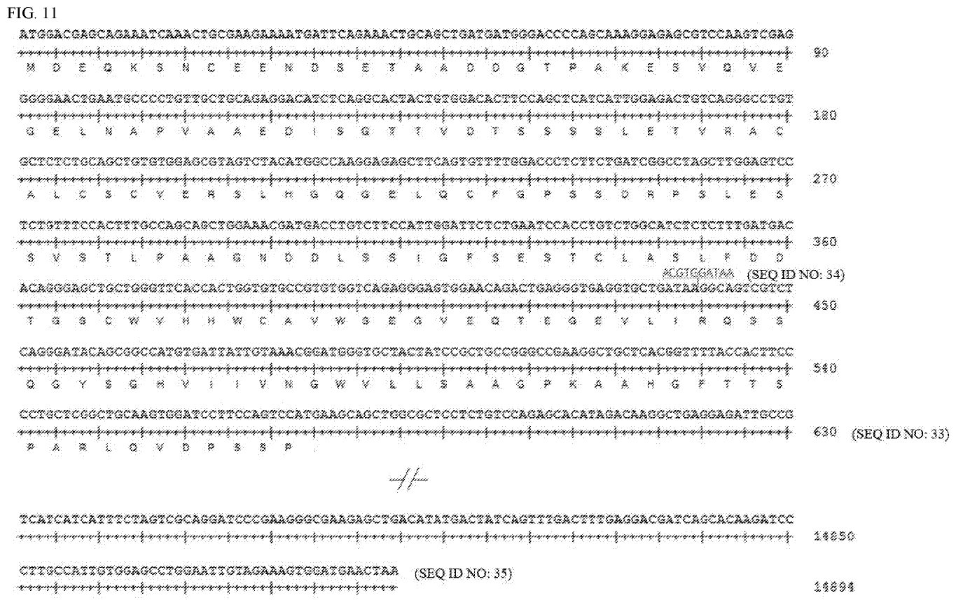

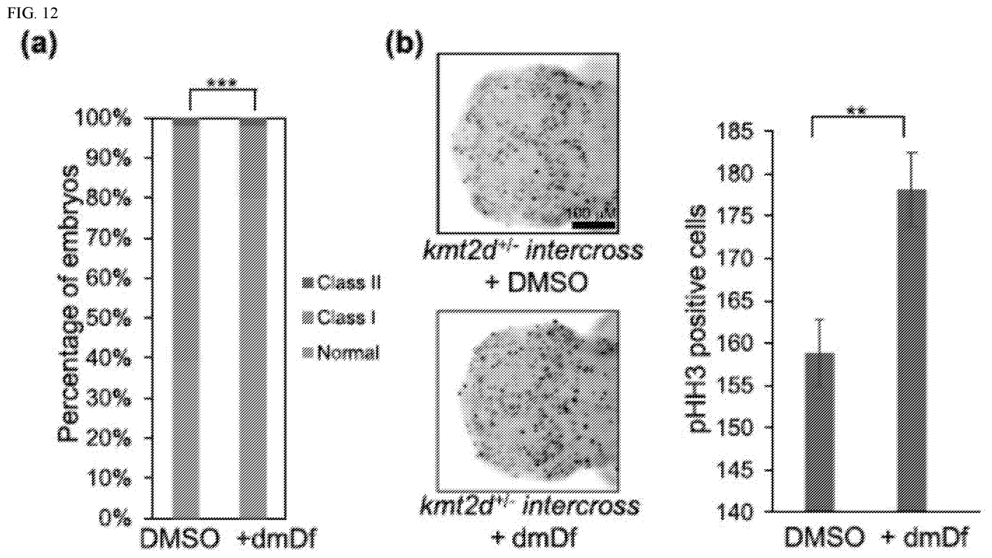

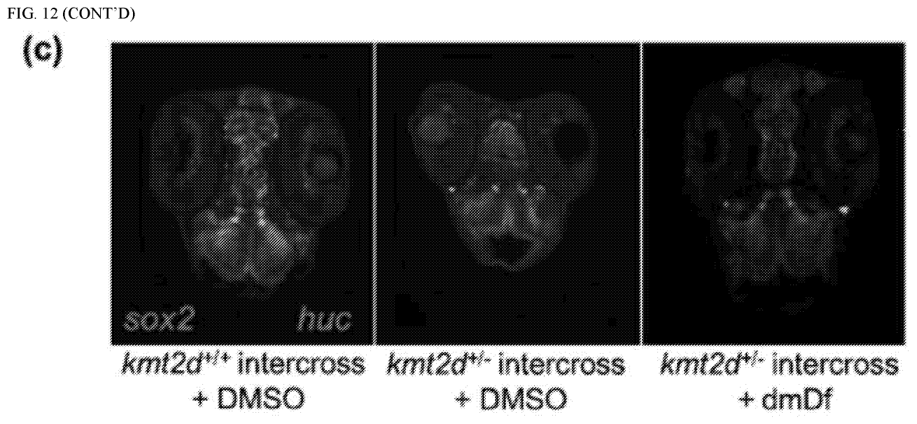

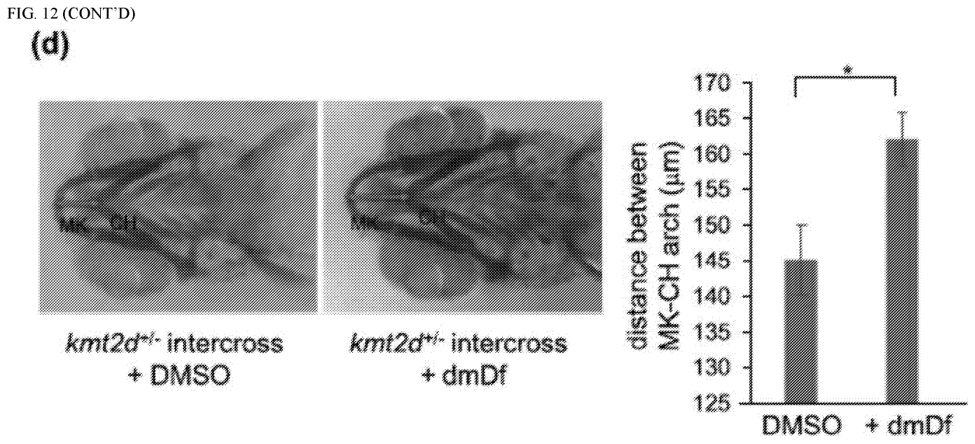

Although the small molecule testing data in CRISPR F0 mosaic models are consistent with morphant phenotypes (and rescue), experiments recognized the necessity of testing the potential efficacy of dmDf in a stable genetic model that is a better genotypic proxy to human patients. Maintaining homozygous zebrafish CRISPR mutants for any KS gene is not possible due to embryonic lethality, a finding consistent with a mouse KS model (see, Bjornsson, H. T. et al. Sci Transl Med 6, 256ra135 (2014)). However, experiments were able to maintain heterozygous kmt2d mutants bearing a 10 bp deletion in exon 6 (kmt2d+/−). This deletion induces a frameshift and premature termination that is predicted to lead to the translation of maximally a 191-residue peptide (instead of a full length 4967-amino acid long protein; FIG. 11). Intercrossing kmt2d+/− and genotyping progeny gave mendelian ratios at 1 dpf (26% wt, 47% −/+, 27% −/−) but showed skewing by 5 dpf (11% −/−), indicative of lethality. Experiments therefore focused the phenotyping efforts in that time window, where intercrosses from F1 progeny were generated, exposed them either to sham or 100 mM dmDf and phenotyped them for CE (1 dpf), neurogenesis (2 dpf) and jaw development (5 dpf). For each phenotype, significant amelioration (averaged measurements across entire zebrafish clutches, replicated) was observed. For CE, in contrast to 60% of embryos showing Class I or Class II defects, dmDf-treated clutches showed defects in only 20% of embryos (p<0.001; FIG. 12A). Similarly, the F1 intercross progeny showed a significant decrease in the number of proliferating cells in the brain, that was likewise rescued by dmDf (FIG. 12B). Finally, given that previous studies have also highlighted neuronal differentiation defects in zebrafish KS model. In addition to examining proliferation of cells in the brain, experiments assessed whether neural differentiation is affected (see, Van Laarhoven, P. M. et al. Hum Mol Genet 24, 4443-53 (2015)). To test this, experiments sectioned 5-10 affected animals at 2 dpf and marked undifferentiated-proliferative cells with sox2 (FIG. 12C) and differentiated/differentiating neural cells with huc ((FIG. 12C). Consistent with the earlier work (see, Van Laarhoven, P. M. et al. Hum Mol Genet 24, 4443-53 (2015)) it was that the organization of both sox2+ and huc+ cells is affected, indicative of perturbed differentiation. However, administration of dmDf attenuated this pathology (FIG. 12C). Finally, such experiments also saw rescue of the mandibular length at 5 dpf (FIG. 12D).

Taken together, these data support the transient MO and F0 rescue data with dmDf and bolster its candidacy as a therapeutic agent.

### Example VIII. Discussion

A persistent issue in the development of therapeutics for rare human genetic disorders is that the ab initio identification of small molecules of therapeutic value faces steep economic and regulatory barriers (see, Sun, P. & Garrison, L. P. Curr Med Res Opin 28, 665-7 (2012)). As such, the community is motivated to look for repurposing opportunities that are guided by biochemical knowledge of disease pathomechanism. Experiments have shown previously that some of the pathognomonic defects of KS are likely driven by the hyperactivation of RAS/MAPK signaling, a pathway studied exhaustively in the context of neoplasia (see, Santarpia, L., Lippman, S. M. & El-Naggar, A. K. Expert Opin Ther Targets 16, 103-19 (2012)). Here, experiments have taken advantage of this overlap to screen a focused set of diverse RAS/MAPK signaling antagonists as a means of providing additional support for a causal link between KS and RAS/MAPK signaling and to explore a possible route to therapeutics. The screening progression path identified six hits covering all three tested RAS-MAPK signaling targets: BRAF, MEK, and ERK. It was found that two compounds were able to repeatedly ameliorate KS-related defects in kmt2d, rap1 and kdm6a zebrafish models. Moreover, at 100 nM, a BRAF antagonist, desmethyl-Dabrafenib, is safe (to the extent that can be tested during zebrafish development) and efficacious at ameliorating all three tested KS-relevant pathologies: convergence and extension during gastrulation, jaw development, and cell proliferation in the developing zebrafish brain. The improvement for the neurological defects particularly draw attention because neurological development in humans continues through childhood and adolescence (see, Stiles, J. & Jernigan, T. L. Neuropsychol Rev 20, 327-48 (2010)). The study reveals the potential of dmDf for the restoration of neurological phenotypes in KS patients by its ability to facilitate brain cell proliferation. However, neurogenesis is a complicated process. A recent study of two KS zebrafish morphants (kmt2d and kdm6a), reported that loss of kmt2d and kdm6a leads to decreased neural cell differentiation (see, Van Laarhoven, P. M. et al. Hum Mol Genet 24, 4443-53 (2015)). Although not a perfect indication for a drug screen, delayed differentiation in our kmt2d mutant was observed; treatment with dmDf ameliorates this pathology. In addition, it was also observed structural heart defects, a phenotype that has been also been reported previously in kmt2d and kdm6a morphants (see, Van Laarhoven, P. M. et al. Hum Mol Genet 24, 4443-53 (2015)). However, the poor dynamic range of this phenotype rendered it intractable to drug screening, while even if efficacious, the clinical application of any small molecule this early in human gestation is likely intractable; as such experiments focused on neurodevelopmental defects.

Retrospectively, the fact that two of 27 compounds showed robust evidence of in vivo rescue at a dose at least an order of magnitude below any observed toxicity supports a pathway-based hypothesis and the potential utility of BRAF or ERK inhibitors for KS. At the same time, it raises the question of why the success rate was not higher. This could be due to differences in absorption efficiency; immature metabolism of zebrafish embryos; or species-specific responses, including differences in target engagement. The fact that the experiments could only detect 250 pM of the most efficacious compound in the exposure experiment (FIG. 5B) suggests that zebrafish rescue assay conditions may not be conducive to high compound exposures. Nonetheless, given the lack of precedence for these kinds of experiments, the inventors were encouraged by the discovery of two compounds, targeting different RAS-MAPK pathway members, that are able to rescue multiple phenotypes in independent zebrafish genetic models of Kabuki syndrome. The rescue of the stable kmt2d mutant was deemed most compelling, since it offers the most control for background uniformity.

Desmethyl-Dabrafenib is a metabolic byproduct of Dabrafenib, a currently prescribed anti-cancer agent, that is generated in the human gut and is either excreted or reabsorbed in the bloodstream, where it builds over time (see, Bershas, D. A. et al. Drug Metab Dispos 41, 2215-24 (2013)). Given that the morphogenesis of the gut is not complete during our investigative window, the lack of efficacy of Dabrafenib is not surprising and might suggest that the metabolic derivative might be the key agent for rescue. Nonetheless, an abundance of caution is warranted. First, given that KS is a pediatric disorder, the tolerance of this population to these compounds is not known. Second, there are fundamental dosing and toxicity questions that must be addressed when one transitions from treating acute somatic disorders to managing chronic germline conditions.

### Example IX. Materials and Methods

### Zebrafish Embryo Manipulation, Microinjection and Compound Treatment.

Morpholinos (MO) were obtained from Gene Tools (Gene Tools, LLC, Philomath, Oreg., USA) and have been described previously (see, Bogershausen, N. et al. J Clin Invest 125, 3585-99 (2015)); kmt2d-MO: 5′-AATCATTTATGTTTACTAACCTGCA-3′(SEQ ID NO: 1) (5 ng); kdm6a-MO: 5′-GGAAACGGACTTTAACTGACCTGTC (SEQ ID NO: 2) (10 ng). kmt2d-MO and kdm6a-MO was injected into wild type (WT) Ekkwill (EK) zebrafish embryos obtained from natural matings at the 1-4 cell stage. CRISPR target sequences (rap1a: 5′-GTGTTGGGCTCTGGTGGTGT-3′ (SEQ ID NO: 3) [120 pg], rap1b: 5′-TGCCAACACCTCCTGATCCG-3′ (SEQ ID NO: 4) [120 pg] (see, Bogershausen, N. et al. J Clin Invest 125, 3585-99 (2015)), and kmt2d: 5′-GGGTGAGGTGCTGATAAACGTGG (SEQ ID NO: 5) [50 pg]) were identified by crispr.mit.edu and cloned into pT7-gRNA (Addgene, Cambridge, USA) following the protocol described in http://www.addgene.org/crispr/Chen/. Guide (g)RNAs were synthesized using the T7 MEGAshortscript kit (ThermoFisher Scientific). An injection solution of gRNA plus 100 pg Cas9 protein (PNA Bio) was injected into wild type Ekkwill (EK) zebrafish embryos obtained from natural matings at the 1-cell stage. Embryos were treated with compound at the 8-cell stage. All compounds were dissolved in dimethyl sulfoxide (DMSO; 10 mM), and subsequently diluted with water to 100 μM. Using egg water, we then diluted the 100 μM solution to the concentration specific to each experiment in egg water. Embryos were incubated at 28.5° C. and egg water containing compound was replaced daily until phenotyping, mass spectrometry, or immunoblotting analysis.

### Zebrafish Embryo and Larval Phenotyping.

For convergence and extension (CE) phenotyping, embryos were scored as described (see, Leitch, C. C. et al. Nat Genet 40, 443-8 (2008)) at the 8-10 somite stage, and images were captured using an AZ100 microscope and NIS Elements software (Nikon). To assess mandibular phenotypes, Alcian blue staining was used to stain cartilage structures in 5 dpf larvae as described (see, Chassaing, N. et al. J Med Genet 49, 373-9 (2012)). Stained larvae were imaged in glycerol using an SMZ745T stereomicroscope (Nikon). The distance between Meckel's (MK) and ceratohyal (CH) cartilages was measured on ventral bright field images with Image J (NIH). For brain cell proliferation assays, injected embryos were fixed in Dent's solution at 2 dpf. Immunohistochemistry was performed with primary antibody for phospho-histone H3 (Santa Cruz Biotechnology; 1:500) and secondary antibody Alexa Fluor goat anti-rabbit IgG (ThermoFisher; 1:1000) according to standard procedures. Fluorescence signal on embryo heads was imaged with the Nikon AZ100 microscope and DS-Qi1MC digital camera using 200 μM Z-stacks to generate extended depth of focus images. Cell numbers were quantified using the ImageJ ICTN plugin (NIH). To assess the neural differentiation, 2 dpf embryos were fixed in 4% PFA overnight, followed by 30% sucrose incubation for another overnight. Embryos were then embedded in O.C.T. (Optimal Cutting Temperature) compound (Tissue-Trek) and sections were cut at 14 μm in a cryostat (LEICA CM3050S). The sections were then stained with sox2 (abcam) and huc (ThermoFisherScientific) antibodies for 1 hr. The images were captured using Nikon 90i microscope. Experiments were repeated at least twice with 30-40 embryos per condition (CE); 30-40 embryos for Alcian blue; and 20 embryos per condition (cell proliferation). Pairwise comparisons to vehicle (DMSO)-treated embryos for rescue efficacy were conducted using a χ2 test (CE) or Student's t-test (mandible and cell proliferation).

Validation of kmt2d-CRISPR Reagents.

To validate the genome-editing efficiency of kmt2d-CRISPR reagents, experiments harvested 10 gRNA/Cas9-injected embryos selected randomly at 1 dpf. Genomic DNA was extracted using 15 μl of 1×Taq buffer (New England Biolabs). The region targeted by kmt2d gRNA was PCR-amplified (Forward: 5′-AAGCAATGGCTATGGTTTGTTTA-3′ (SEQ ID NO: 6) and Reverse: 5′-AAAGGAAGCTCTGTGCCTACC-3′ (SEQ ID NO: 7)). The PCR product was then denatured and re-annealed slowly (95° C. for 5 min, ramping down to 50° C. at 0.1° C./sec, incubating at 50° C. for 10 min, and chilling to 4° C. at 1° C./sec), and subjected to 15% polyacrylamide gel electrophoresis (PAGE) to detect the formation of heteroduplexes as described51. PCR products from six independent embryos were cloned into a pCR4-TOPO vector (Life Technologies), and Sanger sequenced to characterize the changes in the targeted region.

### Western Blot Analysis.

Zebrafish larvae (5 dpf; n=20) were decapitated and heads were homogenized with RIPA buffer (50 mM NaCl, 1% NP40, 50 mM Tris-HCl pH 7.5, 0.1% SDS, 0.5% Na deoxycholate, 1 mM Na3VO4, and 1 mM NaF). Total protein concentration was determined using the BCA Protein Assay Kit (Thermo Fisher Scientific) and 50 μg lysate per condition was subjected to 4-15% SDS-PAGE (Bio-Rad) and transferred to a PVDF membrane. Immunoblots were blocked in 3% BSA in PBS containing 0.1% Tween20 and probed with pMEK1/2 and MEK1/2 (#9121 and #9122, Cell Signaling Technology; 1:2000). Blots were developed using an enhanced chemiluminescence system, Super Signal West Pico Chemiluminescent Substrate Thermo Fisher Scientific), visualized on a ChemiDoc (Bio-Rad) and quantified by Quantity One (Bio-Rad).

### Mass Spectrometry to Determine Compound Uptake in Zebrafish Embryos.

WT EK embryos were transferred to a 12-well plate at the 8-cell stage and bathed in egg water containing 0.001% DMSO or 100 nM of each compound (n=30/condition). Embryos were incubated at 28° C.; egg water containing compound was refreshed every 24 hours until harvest. Compound exposure was assessed essentially as described (see, Berghmans, S. et al. J Pharmacol Toxicol Methods 58, 59-68 (2008)); larvae were anaesthetized on ice, washed once with fresh compound-free ice-cold egg water, washed once with ice-cold PBS, and then transferred to a microfuge tube and centrifuged briefly before removing all excess liquid. Samples were macerated using a plastic pestle and frozen at −20° C. prior to analysis. Mass spectrometry analysis was conducted at Covance Laboratories (Cary, N.C., USA). Briefly, the macerated larvae were diluted in 1 ml water/acetonitrile (50/50), sonicated for 3×10 s and vortexed. For all compounds, a control sample containing embryos exposed to 0.001% DMSO was analyzed as a control. Calibration curves were generated by spiking aliquots of control zebrafish homogenate (prepared as described above) with known amounts of compound 29 (N3521-4-2).

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1-28. (canceled)

29. A method of treating, preventing and/or ameliorating symptoms of a disorder characterized with aberrant RAS/MAPK signaling through administering to a human subject suffering from or at risk of suffering from a disorder characterized with aberrant RAS/MAPK signaling a therapeutically effective amount of an agent capable of one or more of a
inhibiting aberrant RAS/MAPK signaling,
inhibiting MEK hyperactivation related to RAS/MAPK signaling,
inhibiting aberrant kmt2d, kmd6a, RAP1A, and/or RAP1B activity and/or expression related to RAS/MAPK signaling,
preventing diminished RAF1 inhibition of RAP1A or RAP1B activity within RAS/MAPK signaling, and
preventing or ameliorating symptoms of disorders characterized with aberrant RAS/MAPK signaling selected from craniofacial dysmorphisms, musculoskeletal abnormalities, cutaneous abnormalities, cardiac abnormalities, neurocognitive impairment, immune deficits.

30. The method of claim 29, wherein the disorder characterized with aberrant RAS/MAPK signaling is one or more of Kabuki Syndrome, capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, Legius syndrome, and Noonan syndrome with multiple lentigines, and somatic tumors.

31. The method of claim 29, wherein the agent is a pharmaceutical composition comprising a kinase inhibitor that inhibits the activity of BRAF, MEK and/or ERK.

32. The method of claim 31, wherein the agent is 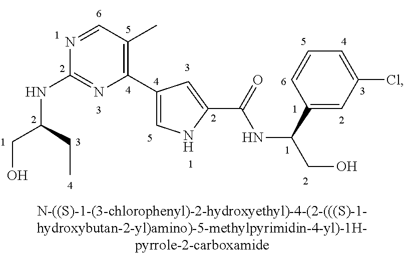 [SMILES: CC[C@@H](CO)Nc1ncc(C)c(-c2c[nH]c(C(=O)N[C@H](CO)c3cccc(Cl)c3)c2)n1]
and any derivatives, salts, and esters thereof, or 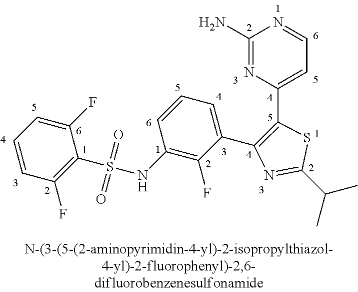 [SMILES: CC(C)c1nc(-c2cccc(NS(=O)(=O)c3c(F)cccc3F)c2F)c(-c2ccnc(N)n2)s1]
(desmethyl-Dabrafenib (dmDf)), and any derivatives, salts, and esters thereof.

33. A method for inhibiting aberrant RAS/MAPK signaling in a subject suffering from or at risk of suffering from a disorder characterized with aberrant RAS/MAPK signaling comprising, consisting of, or consisting essentially of administering to the subject a therapeutically effective amount of a pharmaceutical composition comprising a kinase inhibitor that inhibits the activity of BRAF, MEK and/or ERK.

34. The method of claim 33, wherein the disorder characterized with aberrant RAS/MAPK signaling is one or more of KS, capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, Legius syndrome, Noonan syndrome with multiple lentigines, and somatic tumors.

35. The method of claim 33, wherein the kinase inhibitor is 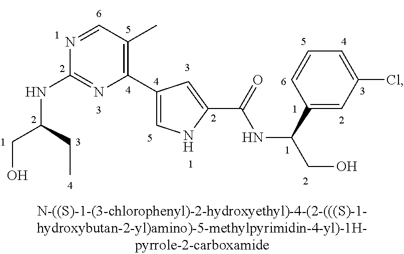 [SMILES: CC[C@@H](CO)Nc1ncc(C)c(-c2c[nH]c(C(=O)N[C@H](CO)c3cccc(Cl)c3)c2)n1]
and any derivatives, salts, and esters thereof, or 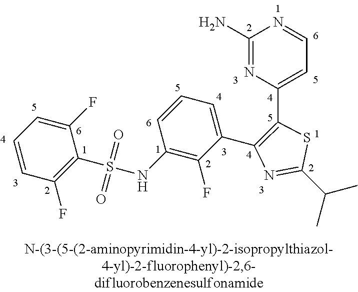 [SMILES: CC(C)c1nc(-c2cccc(NS(=O)(=O)c3c(F)cccc3F)c2F)c(-c2ccnc(N)n2)s1]
(desmethyl-Dabrafenib (dmDf)), and any derivatives, salts, and esters thereof.

36. A method for preventing diminished RAF1 inhibition of RAP1A or RAP1B activity within RAS/MAPK signaling in a subject suffering from or at risk of suffering from a disorder characterized with aberrant RAS/MAPK signaling comprising, consisting of, or consisting essentially of administering to the subject a therapeutically effective amount of a pharmaceutical composition comprising a kinase inhibitor that inhibits the activity of BRAF, MEK and/or ERK.

37. The method of claim 36, wherein the disorder characterized with aberrant RAS/MAPK signaling is one or more of KS, capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, Legius syndrome, Noonan syndrome with multiple lentigines, and somatic tumors.

38. The method of claim 37, wherein the kinase inhibitor is 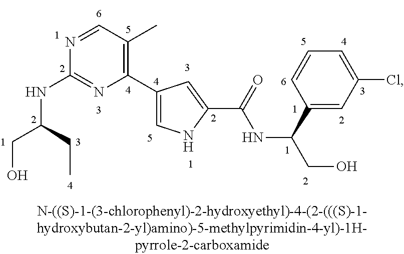 [SMILES: CC[C@@H](CO)Nc1ncc(C)c(-c2c[nH]c(C(=O)N[C@H](CO)c3cccc(Cl)c3)c2)n1]
and any derivatives, salts, and esters thereof, or 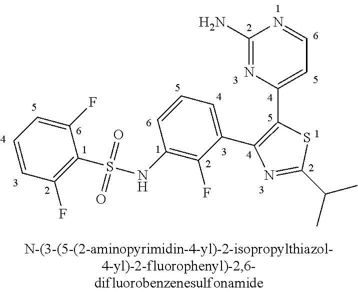 [SMILES: CC(C)c1nc(-c2cccc(NS(=O)(=O)c3c(F)cccc3F)c2F)c(-c2ccnc(N)n2)s1]
(desmethyl-Dabrafenib (dmDf)), and any derivatives, salts, and esters thereof.


SEQUENCE LISTING (35 sequences)

SEQ ID NO: 1 - DNA, 25 bp, Artificial Sequence
  Synthetic
aatcatttatgtttactaacctgca

SEQ ID NO: 2 - DNA, 25 bp, Artificial Sequence
  Synthetic
ggaaacggactttaactgacctgtc

SEQ ID NO: 3 - DNA, 20 bp, Artificial Sequence
  Synthetic
gtgttgggctctggtggtgt

SEQ ID NO: 4 - DNA, 20 bp, Artificial Sequence
  Synthetic
tgccaacacctcctgatccg

SEQ ID NO: 5 - DNA, 23 bp, Artificial Sequence
  Synthetic
gggtgaggtgctgataaacgtgg

SEQ ID NO: 6 - DNA, 23 bp, Artificial Sequence
  Synthetic
aagcaatggctatggtttgttta

SEQ ID NO: 7 - DNA, 21 bp, Artificial Sequence
  Synthetic
aaaggaagctctgtgcctacc

SEQ ID NO: 8 - DNA, 76 bp, Artificial Sequence
  Synthetic
gtgtgccgtgtggtcagagggagtggaacagactgagggtgaggtgctgataaacgtggataaggcagtcgtctca

SEQ ID NO: 9 - DNA, 66 bp, Artificial Sequence
  Synthetic
gtgtgccgtgtggtcagagggagtggaacagactgagggtgaggtgctgataaggcagtcgtctca

SEQ ID NO: 10 - DNA, 65 bp, Artificial Sequence
  Synthetic
gtgtgccgtgtggtcagagggagtggaacagatgagggtgaggtgctgataaggcagtcgtctca

SEQ ID NO: 11 - DNA, 66 bp, Artificial Sequence; identical to SEQ ID NO: 9
  Synthetic

SEQ ID NO: 12 - DNA, 18 bp, Artificial Sequence
  Synthetic
gataaggcagtcgtctca

SEQ ID NO: 13 - DNA, 77 bp, Artificial Sequence
  Synthetic
gtgtgccgtgtggtcagagggagtggaacagactgagggtgaggtgctgataaaacgtggataaggcagtcgtctca

SEQ ID NO: 14 - DNA, 77 bp, Artificial Sequence
  Synthetic
gtgtgccgtgtggtcagagggagtggaacagactgagggtgaggtgctgataaggcatggataaggcagtcgtctca

SEQ ID NO: 15 - DNA, 74 bp, Artificial Sequence
  Synthetic
gtgtgccgtgtggtcagagggagtggaacagactgagggtgaggtgctgataagactgttaggcagtcgtctca

SEQ ID NO: 16 - DNA, 60 bp, Artificial Sequence
  Synthetic
gtgtgccgtgtggtcagagggagtggaacagactgagggtgagataaggcagtcgtctca

SEQ ID NO: 17 - DNA, 66 bp, Artificial Sequence; identical to SEQ ID NO: 9
  Synthetic

SEQ ID NO: 18 - DNA, 64 bp, Artificial Sequence
  Synthetic
gtgtgccgtgtggtcagagggagtggaacagactgagggtgacgtggataaggcagtcgtctca

SEQ ID NO: 19 - DNA, 66 bp, Artificial Sequence; identical to SEQ ID NO: 9
  Synthetic

SEQ ID NO: 20 - DNA, 67 bp, Artificial Sequence
  Synthetic
gtgtgccgtgtggtcagagggagtggaacagactgagggtgaggtgctgataaggcagtcgttctca

SEQ ID NO: 21 - DNA, 77 bp, Artificial Sequence; identical to SEQ ID NO: 13
  Synthetic

SEQ ID NO: 22 - DNA, 66 bp, Artificial Sequence; identical to SEQ ID NO: 9
  Synthetic

SEQ ID NO: 23 - DNA, 74 bp, Artificial Sequence
  Synthetic
gtgtgccgtgtggtcagagggagtggaacagactgagggtgaggtgctgatacgtggataaggcagtcgtctca

SEQ ID NO: 24 - DNA, 73 bp, Artificial Sequence
  Synthetic
gtgtgccgtgtggtcagagggagtggaacagactgagggtgaggtgctgaacgtggataaggcagtcgtctca

SEQ ID NO: 25 - DNA, 68 bp, Artificial Sequence
  Synthetic
gtgtgccgtgtggtcagagggagtggaacagactgagggtgaggtgcgtggataaggcagtcgtctca

SEQ ID NO: 26 - DNA, 76 bp, Artificial Sequence
  Synthetic
gtgtgccgtgtggtcagagggagtggaacagactgagggtgaggtgctgataaggcaggatacggcagtcgtctca

SEQ ID NO: 27 - DNA, 66 bp, Artificial Sequence; identical to SEQ ID NO: 9
  Synthetic

SEQ ID NO: 28 - DNA, 74 bp, Artificial Sequence; identical to SEQ ID NO: 23
  Synthetic

SEQ ID NO: 29 - DNA, 77 bp, Artificial Sequence; identical to SEQ ID NO: 13
  Synthetic

SEQ ID NO: 30 - DNA, 67 bp, Artificial Sequence
  Synthetic
gtgtgccgtgtggtcaagagggagtggaacagactgagggtgaggtgctgataaggcagtcgtctca

SEQ ID NO: 31 - DNA, 66 bp, Artificial Sequence; identical to SEQ ID NO: 9
  Synthetic

SEQ ID NO: 32 - DNA, 69 bp, Artificial Sequence
  Synthetic
gtgtgccgtgtggtgcagagggagtggaacagactgagggtgaggtgcgtggataaggcagtcgtctca

SEQ ID NO: 33 - DNA, 630 bp, Artificial Sequence
  Synthetic
atggacgagcagaaatcaaactgcgaagaaaatgattcagaaactgcagctgatgatgggaccccagcaaaggagagcgtccaagtcgagggggaactgaatgcccctgttgctgcagaggacatctcaggcactactgtggacacttccagctcatcattggagactgtcagggcctgtgctctctgcagctgtgtggagcgtagtctacatggccaaggagagcttcagtgttttggaccctcttctgatcggcctagcttggagtcctctgtttccactttgccagcagctggaaacgatgacctgtcttccattggattctctgaatccacctgtctggcatctctctttgatgacacagggagctgctgggttcaccactggtgtgccgtgtggtcagagggagtggaacagactgagggtgaggtgctgataaggcagtcgtctcagggatacagcggccatgtgattattgtaaacggatgggtgctactatccgctgccgggccgaaggctgctcacggttttaccacttcccctgctcggctgcaagtggatccttccagtccatgaagcagctggcgctcctctgtccagagcacatagacaaggctgaggagattgccg

SEQ ID NO: 34 - DNA, 632 bp, Artificial Sequence
  Synthetic
atggacgagcagaaatcaaactgcgaagaaaatgattcagaaactgcagctgatgatgggaccccagcaaaggagagcgtccaagtcgagggggaactgaatgcccctgttgctgcagaggacatctcaggcactactgtggacacttccagctcatcattggagactgtcagggcctgtgctctctgcagctgtgtggagcgtagtctacatggccaaggagagcttcagtgttttggaccctcttctgatcggcctagcttggagtcctctgtttccactttgccagcagctggaaacgatgacctgtcttccattggattctctgaatccacctgtctggcatctctctttgatgacacagggagctgctgggttcaccactggtgtgccgtgtggtcagagggagtggaacagactgagggtgaggtgctgacgtggataagtcgtctcagggatacagcggccatgtgattattgtaaacggatgggtgctactatccgctgccgggccgaaggctgctcacggttttaccacttcccctgctcggctgcaagtggatccttccagtccatgaagcagctggcgctcctctgtccagagcacatagacaaggctgaggagattgccg

SEQ ID NO: 35 - DNA, 134 bp, Artificial Sequence
  Synthetic
tcatcatcatttctagtcgcaggatcccgaagggcgaagagctgacatatgactatcagtttgactttgaggacgatcagcacaagatcccttgccattgtggagcctggaattgtagaaagtggatgaactaa